(19) United States
(12) Patent Application Publication
(10) Pub. No.: US 2023/0313146 A1
(43) Pub. Date: Oct. 5, 2023

(54) INTESTINAL EPITHELIAL CELL CULTURES

(71) Applicant: ARDELYX, INC., Waltham, MA (US)

(72) Inventor: Matthew John Siegel, Waltham, MA (US)

(21) Appl. No.: 18/132,215

(22) Filed: Apr. 7, 2023

Related U.S. Application Data
(63) Continuation-in-part of application No. 16/953,012, filed on Nov. 19, 2020.
Application No. 16/953,012 is a continuation-in-part of application No. 15/971,683, filed on May 4, 2018, now abandoned.
(60) Provisional application No. 62/501,307, filed on May 4, 2017.

(51) Int. Cl. C12N 5/071

(52) U.S. Cl. CPC C12N 5/0679; C12N 2501/11; C12N 2501/155

(57) ABSTRACT

Provided are compositions and methods for generating two-dimensional (2D) intestinal epithelial cell cultures for all segments of mouse and human small and large intestines. The compositions and methods described herein utilize primary human or murine intestinal cells and do not rely on cancer cell lines, resulting in 2D cultures that are representative of homeostatic epithelial gene expression and function. Also provided are compositions and methods of utilizing the cultures described herein in a high-throughput system for compound evaluation.

### CROSS-REFERENCE TO RELATED APPLICATIONS

This application is a continuation-in-part of U.S. patent application Ser. No. 16/953,012, filed Nov. 19, 2020, which is a continuation-in-part of U.S. patent application Ser. No. 15/971,683, filed May 4, 2018, and claims priority to U.S. provisional application Ser. No. 62/501,307, filed May 4, 2017, the contents of each of which are incorporated herein by reference in their entirety.

### BACKGROUND

The intestinal epithelium is composed of a single layer of polarized columnar epithelial cells with a diverse range of functions. These functions include acting as a barrier to separate the intestinal luminal contents and microbes from the underlying tissue, controlling the absorption of nutrients and the excretion of waste, secreting hormones for paracrine and endocrine signaling, and communicating with the underlying gut-associated lymphoid tissue (Furness et al., (1999), Am J Physiol 277(5 Pt 1):G922-928). The varied gene expression along the longitudinal axis of the intestine highlights the multifunctional role of intestinal epithelial cells. For example, the abundance of bicarbonate transporters expressed in the duodenum is consistent with the role of the duodenum in the neutralization of acidic chime that enters from the stomach (Seidler U, et al. (2011) Acta Physiol (Oxf) 201(1):3-20). Further, the jejunum expresses an abundance of digestive brush border enzymes and nutrient transporters, consistent with its role in nutrient digestion and assimilation; the large number of bile acid transporters in the ileum facilitate bile acid reabsorption; and the many electrolyte transporters in the colon facilitate water and electrolyte reabsorption in the colon (Daniel H (2004) Annu Rev Physiol 66:361-384; Dawson et al., (2009) J Lipid Res 50(12):2340-2357; Iqbal et al., (2009) Am J Physiol Endocrinol Metab 296(6):E1183-1194; Kunzelmann et al., (2002) Physiol Rev 82(1):245-289; Takata et al., (1992) Cell Tissue Res 267(1):3-9).

Minimally systemic drugs that target and modulate intestinal epithelial function exert their pharmacological activity with little or no systemic absorption, thereby minimizing unnecessary exposure of other organ systems to the drug and reducing the potential for side effects (Charmot D, (2012), Curr Pharm Des 18(10):1434-1445). In vitro screening systems facilitate the expedited development of these drugs, enabling quick assessment of the specificity and particular physiologic effects of a large number of small molecules in a high-throughput system. However, these large-scale screens are difficult to perform on human intestinal epithelial cells due to the limited amount of accessible primary tissue. As such, initial studies often use intestinal cancer cell lines, such as Caco-2, and murine intestinal cells, due to the relative ease of culture and abundance of tissue. However, these systems pose many limitations. For example, cancer cell lines contain known and unknown mutations and which may not accurately represent any one segment or cell type of the gut (Delie et al., (1997), Crit Rev Ther Drug Carrier Syst 14(3):221-286; Sun et al., (2008) Expert Opin Drug Metab Toxicol 4(4):395-411). Further, although there are a number of overlapping genes and functional characteristics between murine and human intestinal epithelial cells, assessing the function of a particular compound on a human cell is a critical aspect of drug development.

Current methods for in vitro drug screen have used intestinal organoid and enteroid culture systems as a more physiologically relevant culture system. Intestinal organoids are three-dimensional intestinal epithelial cell cultures and have been shown to recapitulate gastrointestinal epithelial cell biology in many aspects (e.g., gene expression and general cellular function) and have provided an invaluable resource for exploring fundamental intestinal cellular and molecular biology (Farin H F, et al. (2014), J Exp Med 211(7):1393-1405; Grun D, et al. (2015), Nature 525(7568):251-255; Farin H F, et al. (2016), Nature 530(7590):340-343; Middendorp S, et al. (2014), Stem Cells 32(5):1083-1091). Organoid cultures from the mouse small intestine bud crypt-like structures containing intestinal stem cells, Paneth cells, and transit-amplifying cells, while cells close to the pseudo-lumen differentiate into absorptive and secretory cell lineages (Sato T, et al. (2009), Nature 459(7244):262-265). Further, cells derived from intestinal organoids can be transplanted in vivo, where they are able to integrate into intestinal tissue in the absence functional or histological abnormalities (Yui S, et al. (2012), Nat Med 18(4):618-623). However, use of intestinal organoids in the development of functional assays to screen pharmacological compounds has remained limited (Dekkers J F, et al. (2013), Nat Med 19(7):939-945; van de Wetering M, et al. (2015), Cell 161(4):933-945).

A distinct limitation of three-dimensional organoids is that the apical membrane faces the inside of the organoid, requiring micro-injection for transport studies and resulting in the accumulation of dead cells and mucus inside the pseudo-lumen. In order to make intestinal organoids more suitable for assay development and functional evaluation, intestinal epithelial organoid cultures can be converted into monolayer cultures. In monolayer cultures, cells develop many of the hallmarks of endogenous intestinal epithelial cells including polarized membranes, formation of tight junctions, and differentiation into the major intestinal cell lineages the apical and basolateral membranes are easily accessible. Further, the two-dimensional nature of the culture system enables easy access to the apical and basolateral membranes.

However, intestinal epithelial cells derived from different locations within the intestine (e.g., small vs. large intestine) exhibit distinct gene expression patterns and functional characteristics. Further, the precise culturing methods required for effective and reliable generation of differentiated monolayer cultures that are representative of these varied cell types and functions have not been described. As such, there is a need in the art for culture protocols for both human and murine intestinal epithelial cells that enable the generation of differentiated, polarized cultures representing the endogenous physiology of intestinal epithelial cells in order to accelerate the discovery and development of minimally systemic drugs. Such systems allow for structure-activity relationship optimization, mechanism-of-action studies, new target identification, and drug permeability measurements in the native cell type expressing native drug targets. Moreover, development of coordinated rodent and human intestinal epithelial cell cultures to evaluate drug candidates would minimize translational risks in initiating clinical studies.

### BRIEF SUMMARY

The present disclosure provides for methods of generating two-dimensional (2D) monolayer intestinal epithelial cell cultures for all segments of the mouse and human small and large intestines. The methods described herein utilize primary human or murine intestinal cells and do not rely on cancer cell lines, resulting in 2D cultures that are representative of homeostatic epithelial gene expression and function. In some embodiments, the present disclosure provides methods of utilizing the cultures described herein in a high-throughput system for compound evaluation. Therefore, in some embodiments, the present disclosure provides culturing and screening methods for use in drug discovery platforms.

Embodiments of the present disclosure include methods for generating a two-dimensional (2D) monolayer cell culture of primary intestinal cells comprising the steps of: (a) isolating cells from a mammalian tissue sample, wherein the tissue sample is a small intestine or a colon tissue sample; (b) plating the cells in a monolayer in a well in the presence of a seeding medium, wherein the seeding medium comprises epidermal growth factor (EGF), a bone morphogenic protein (BMP) inhibitor, a leucine-rich repeat-containing G-protein coupled receptor (LGR)-5 activator, a Wnt signaling agonist, and a Rho-associated protein kinase (ROCK) inhibitor; (c) growing the cells to a confluent monolayer in a growth medium; and (d) differentiating the cells in a differentiation medium for a time sufficient for the cells to develop mature phenotype(s); thereby generating a 2D monolayer cell culture of primary intestinal cells. In certain embodiments, the primary intestinal cells comprise one or more of enterocytes, goblet cells, enteroendocrine cells, Paneth cells, transit amplifying cells, and stem cells.

In certain embodiments, the seeding medium, growth medium, and/or differentiation medium further comprise a growth promoting and/or an antioxidant factor. In certain embodiments, the growth promoting factor comprises an N2 or B27 supplement. In certain embodiments, the antioxidant factor comprises N-acetylcysteine. In certain embodiments, the seeding medium comprises a concentration of about 5-500 ng/mL of EGF. In certain embodiments, the BMP inhibitor is Noggin and is at a concentration of about 10 ng/mL to about 500 ng/mL. In certain embodiments, the LGR5 activator is R-spondin 1 and is at a concentration of about 50 ng/mL to about 2 μg/mL, or about 100 ng/mL to about 1000 ng/mL. In certain embodiments, the Wnt signaling agonist is Wnt3a and is at a concentration of about 20 ng/mL to about 1 μg/mL. In certain embodiments, the ROCK inhibitor is Y-27632 or thiazovivin. In certain embodiments, the seeding medium comprises a Y-27632 concentration of about 1 μM to about 100 μM, or a thiazovivin concentration of about 0.5 μM to about 25 μM. In certain embodiments, the seeding medium comprises B27, N2, N-acetylcysteine, EGF, Noggin, R-Spondin-1, Wnt3a, and Y-27632. In certain embodiments, the seeding medium comprises B27, N2, about 1 mM N-acetylcysteine, about 50 ng/mL EGF, about 0.1 μg/mL Noggin, about 250 ng/mL Wnt3a, about 0.5 μg/mL R-spondin 1, and about 20 μM Y27632. In certain embodiments, the growth medium comprises EGF, a BMP inhibitor, an LGR5 activator, and a Wnt signaling agonist. In certain embodiments, the concentration of EGF is about 5-500 ng/mL. In certain embodiments, the BMP inhibitor is Noggin and is at a concentration of about 10 ng/mL to about 500 ng/mL. In certain embodiments, the LGR5 activator is R-spondin 1 and is at a concentration of about 50 ng/mL to about 2 μg/mL. In certain embodiments, the Wnt signaling agonist is WNT3a and is at a concentration of about 20 ng/mL to about 1 μg/mL, or about 100 ng/mL to about 1000 ng/mL. In certain embodiments, the growth medium comprises N2, B27, N-acetylcysteine, EGF, Noggin, R-spondin1, and Wnt3a. In certain embodiments, the growth medium comprises N2, B27, about 1 mM N-acetylcysteine, about 50 ng/mL EGF, about 0.1 μg/mL Noggin, about 0.5 μg/mL R-spondin1, and about 250 ng/mL Wnt3a. In certain embodiments, the growth medium further comprises a ROCK inhibitor. In certain embodiments, the ROCK inhibitor is selected from Y-27632 and thiazovivin. In certain embodiments, the growth medium comprises a Y-27632 concentration of about 1 μM to about 100 μM, or a thiazovivin concentration of about 0.5 μM to about 25 μM. In certain embodiments, the growth medium comprises N2, B27, N-acetylcysteine, EGF, Noggin, R-spondin1, Wnt3a, and Y-27632. In certain embodiments, the growth medium comprises N2, B27, about 1 mM N-acetylcysteine, about 50 ng/mL EGF, about 0.1 μg/mL Noggin, about 0.5 μg/mL R-spondin1, about 250 ng/mL Wnt3a, and about 20 μM Y-27632.

In certain embodiments, the differentiation medium comprises EGF and does not comprise detectable amounts of R-spondin 1 or Wnt3a. In certain embodiments, the differentiation medium further comprises a BMP inhibitor. In certain embodiments, the BMP inhibitor comprises Noggin at a concentration of about 10 ng/mL to about 500 ng/mL. In certain embodiments, the differentiation medium comprises N2, B27, N-acetylcysteine, EGF, and Noggin. In certain embodiments, the differentiation medium comprises N2, B27, about 1 mM N-acetylcysteine, about 50 ng/mL EGF, and about 0.1 μg/mL Noggin. In certain embodiments, the differentiation medium further comprises a ROCK inhibitor and a growth factor that promotes cell differentiation. In certain embodiments, the growth factor is a bone morphogenetic protein (BMP). In certain embodiments, the BMP is selected from BMP2, BMP4, BMP7, and any combination thereof. In certain embodiments, the BMP is at a concentration of about 10 ng/mL to about 1000 ng/mL. In certain embodiments, the ROCK inhibitor is Y-27632 at a concentration of about 1 μM to about 100 μM, or thiazovivin at a concentration of about 0.5 μM to about 25 μM. In certain embodiments, the differentiation medium comprises N2, B27, N-acetylcysteine, EGF, thiazovivin, and BMP4. In certain embodiments, the differentiation medium comprises N2, B27, about 1 mM N-acetylcysteine, about 50 ng/mL EGF, about 2.5 μM thiazovivin, and about 100-300 ng/mL BMP4.

In certain embodiments, the seeding medium is incubated with the cells between 1-3 days after plating, optionally 2 days after plating the cells. In certain embodiments, the growth medium is added on days 2 and 4 after plating the cells or on days 1 and 3 after plating the cells. In certain embodiments, the differentiation medium is added between 4-6 days after plating the cells, optionally 5 days after plating the cells. In certain embodiments, the differentiation medium is added when the cultures have a transepithelial electrical resistance (TEER) value greater than 100 Ω·cm². In certain embodiments, the cultures are incubated in the differentiation medium for 1-4 days, optionally 2-3 days, to allow for cell differentiation. In certain embodiments, the cultures have a TEER value greater than or equal to 250 Ω·cm² 1-4 days after incubation in the differentiation medium, optionally 2-3 days after incubation in the differentiation medium.

In certain embodiments, the mammalian tissue sample is a murine tissue sample. In certain embodiments, the murine tissue sample is a small intestine tissue sample comprising tissue from one or more of the duodenum, jejunum, or ileum. In certain embodiments, the murine tissue sample is a colon tissue sample. In certain embodiments, isolating cells from a mammalian tissue sample comprises: (i) isolating crypts from a mammalian tissue sample; and (ii) dissociating cells from the crypts. In certain embodiments, isolating crypts from a mammalian tissue sample comprises: incubating the sample in a first isolation buffer at about 4° C.; incubating the sample in a second isolation buffer at about 37° C.; and—applying force to the sample through shaking, mixing, triturating, or other means.

In certain embodiments, the first isolation buffer comprises PBS, EDTA, and DTT. In certain embodiments, the second isolation buffer comprises PBS and EDTA.

In certain embodiments, dissociating cells from the crypts comprises: —contacting the crypts with a dissociation medium; —incubating the crypts at about 37° C.; and —intermittently applying force to the crypts through shaking, mixing, triturating, or other means.

In certain embodiments, the dissociation medium comprises dispase or TrypLE Express.

Also included are methods for generating a two-dimensional (2D) monolayer cell culture of stable, primary intestinal cells comprising: (a) obtaining organoids, wherein the organoids are human intestinal organoids cultured from a human small intestine tissue sample or a human colon tissue sample; (b) dissociating cells from the organoids; (c) plating the cells in a monolayer in a well in the presence of a seeding culture medium, wherein the seeding culture medium comprises epidermal growth factor (EGF), a bone morphogenic protein (BMP) inhibitor, a leucine-rich repeat-containing G-protein coupled receptor (LGR)-5 activator, a Wnt signaling agonist, a transforming growth factor (TGF)-β signaling antagonist, and a ROCK inhibitor; (d) growing the cells to a confluent monolayer in a growth medium; and (e) differentiating cells in a differentiation medium for a time sufficient for the cells to develop mature phenotype(s).

In certain embodiments, obtaining organoids of step (a) comprises: (i) isolating crypts and dissociated cells from a human intestinal tissue sample; and (ii) incubating the crypts or dissociated cells under three-dimensional (3D) organoid growth conditions in an extracellular matrix in the presence of an organoid culture medium for a time sufficient for the crypts and/or dissociated cells to grow into organoids, wherein the human intestinal tissue sample is from a small intestine or a colon, and wherein the organoid culture medium comprises EGF, a BMP inhibitor, an LGR5 activator, a Wnt signaling agonist, a TGFβ signaling antagonist, a p38 mitogen activated kinase inhibitor, nicotinamide and a ROCK inhibitor.

In certain embodiments, isolating crypts and dissociated cells from a human intestinal tissue sample comprises: placing the sample in a first dissociation buffer comprising a protease; and dissociating the sample by force through shaking, mixing, triturating, or other means.

In certain embodiments, dissociating cells from the organoids comprises: (i) placing the organoids in a second dissociation buffer comprising a protease; and (ii) dissociating the organoids by force through shaking, mixing, triturating, or other means.

In certain embodiments, the protease is a commercially available protease. In certain embodiments, the primary intestinal cells comprise one or more of enterocytes, goblet cells, enteroendocrine cells, Paneth cells, transit amplifying cells, and stem cells. In certain embodiments, the seeding medium, growth medium, differentiation medium, and/or the organoid culture medium comprise a growth promoting factor and/or an antioxidant factor. In certain embodiments, the growth promoting factor comprises N2, B27, and/or a gastrin analog. In certain embodiments, the antioxidant factor comprises N-acetylcysteine. In certain embodiments, the seeding medium comprises a concentration about 5-500 ng/mL of EGF. In certain embodiments, the BMP inhibitor is Noggin at a concentration of about 10 ng/mL to about 500 ng/mL. In certain embodiments, the LGR5 activator is R-spondin 1 at a concentration of about 50 ng/mL to 2 μg/mL. In certain embodiments, the Wnt signaling agonist is Wnt3a at a concentration of about 20 ng/mL to about 1 μg/mL or about 100 ng/mL to about 1000 ng/mL. In certain embodiments, the TGFβ signaling antagonist is selected from one or more of A83-01, GW788388, LY364947, R268712, RepSox, SB431542, SB505124, and SB525334. In certain embodiments, the seeding medium comprises a concentration of about 100 nM to 2000 nM of A83-01. In certain embodiments, the ROCK inhibitor is selected from thiazovivin or Y-27632. In certain embodiments, the ROCK inhibitor is thiazovivin at a concentration of about 0.5 μM to about 25 μM. In certain embodiments, the seeding medium comprises N2, B27, N-acetylcysteine, Leu-15 gastrin, EGF, Noggin, R-spondin1, Wnt3a, A83-01, and thiazovivin. In certain embodiments, the seeding medium comprises N2, B27, about 1 mM N-acetylcysteine, about 10 nM Leu-15 gastrin, about 50 ng/mL EGF, about 1 μg/mL Noggin, about 0.5 μg/mL R-spondin1, about 250 ng/mLWnt3a, about 0.5 μM A83-01, and about 2.5 μM thiazovivin. In certain embodiments, the ROCK inhibitor is Y-27632 at a concentration of about 1 μM to about 100 μM. In certain embodiments, the seeding medium comprises N2, B27, N-acetylcysteine, Leu-15 gastrin, EGF, Noggin, R-spondin1, Wnt3a, A83-01, and Y-27632. In certain embodiments, the seeding medium comprises N2, B27, about 1 mM N-acetylcysteine, about 10 nM Leu-15 gastrin, about 50 ng/mL EGF, about 1 μg/mL Noggin, about 0.5 μg/mL R-spondin1, about 250 ng/mLWnt3a, about 0.5 μM A83-01, and about 20 μM Y-27632. In certain embodiments, the seeding medium and growth medium are substantially similar. In certain embodiments, the differentiation medium comprises EGF, a BMP inhibitor, and a TGFβ signaling antagonist, and wherein the differentiation medium does not comprise detectable amounts of Wnt3a. In certain embodiments, the BMP inhibitor is Noggin and is at a concentration of about 10 ng/mL to about 500 ng/mL. In certain embodiments, the TGFβ signaling antagonist is selected from one or more of A83-01, GW788388, LY364947, R268712, RepSox, SB431542, SB505124, and SB525334. In certain embodiments, differentiation medium comprises a concentration of about 100 nM to 2000 nM of A83-01. In certain embodiments, the differentiation medium comprises N2, B27, N-acetylcysteine, Leu-15 gastrin, EGF, Noggin, and A83-01. In certain embodiments, the differentiation medium comprises N2, B27, about 1 mM N-acetylcysteine, about 10 nM Leu-15 gastrin, about 50 ng/mL EGF, about 1 μg/mL Noggin, and about 0.5 μM A83-01. In certain embodiments, the differentiation medium further comprises an LGR5 activator. In certain embodiments, the LGR5 activator is R-spondin 1 and is at a concentration of about 50 ng/mL to about 2 μg/mL. In certain embodiments, the differentiation medium comprises N2, B27, N-acetylcysteine, Leu-15 gastrin, EGF, Noggin, R-spondin 1, and A83-01. In certain embodiments, the differentiation medium comprises N2, B27, about 1 mM N-acetylcysteine, about 10 nM Leu-15 gastrin, about 50 ng/mL EGF, about 1 μg/mL Noggin, 0.5 μg/mL R-spondin 1, and about 0.5 μM A83-01.

In certain embodiments, step (ii) comprises the steps of: embedding the crypts or dissociated cells in a first solution comprising extracellular matrix proteins; allowing the extracellular matrix to solidify at about 37° C.; and contacting the crypts or dissociated cells and the extracellular matrix with the organoid culture medium.

In certain embodiments, the first solution comprises a concentration of about 1-20 mg/mL extracellular matrix proteins. In certain embodiments, the extracellular matrix proteins comprise Matrigel. Some embodiments include embedding the crypts or dissociated cells are embedded into the first solution at a density of about 1-500 cells/μL. Some embodiments include incubating the crypts or dissociated cells for about 3 days to 3 weeks in organoid culture medium.

In certain embodiments, step (b) comprises dissociating the organoid with a protease. In certain embodiments, the protease has trypsin-like activity. In certain embodiments, the protease is TrypLE Express.

In certain embodiments, the seeding culture medium is incubated with the cells for between 1-6 days, optionally between 2-5 days, or optionally for 3 days. In certain embodiments, the cultures have transepithelial electrical resistance values greater than 100 Ω·cm² three days after seeding the cells. In certain embodiments, the differentiation medium is applied to the cells for between 1-5 days, optionally between 2-4 days, or optionally for 3 days to allow cells to differentiate. In certain embodiments, the cultures have transepithelial electrical resistance measurements of greater than 250 Ω·cm² between 1-5 days, between 2-4 days, or 3 days after addition of cell differentiation medium. In certain embodiments, the human tissue sample is from a biopsy. In certain embodiments, the organoids are enteroids and the human tissue sample is from a biopsy of the duodenum, jejunum, or ileum. In certain embodiments, the organoids are colonoids and the human tissue sample is a colon sample.

In certain embodiments, the cells are plated in a well comprising one or more extracellular matrix proteins, and wherein the well is coated with the one or more extracellular cellular matrix proteins by: (i) contacting the well with a solution comprising 0.1 mg/mL-5 mg/mL of the one or more extracellular matrix proteins in an amount of about 50-1000 μl of solution per cm² of well surface area; (ii) incubating the solution and the well at about 4° C. to 37° C.; and (iii) removing the solution from the well. In certain embodiments, the solution comprises a concentration of about 0.1-2 mg/mL extracellular matrix proteins, optionally wherein the concentration is about 0.4 mg/mL extracellular matrix proteins. In certain embodiments, the well is contacted with the solution in an amount of 300 μL of solution per cm² of well surface area. In certain embodiments, the solution and the well are incubated at ambient temperature. In certain embodiments, the cells are plated at a density of about 10⁴-10⁶ cells/cm², optionally wherein the density is 3×10⁵ cells/cm². In certain embodiments, the well is a standard well or a transwell. In certain embodiments, the transwell is on a multiple-well plate selected from a 24-well plate, a 96-well plate, a 384-well plate, and a 1536-well plate.

Also included are methods of screening an agent in a primary cell culture obtained by the methods described herein, comprising: (a) obtaining a first well comprising the primary cells in the presence of a cell culture medium comprising EGF, a BMP inhibitor, and optionally an LGR5 activator and a Wnt signaling agonist, wherein the primary cells originate from small intestine or colon; (b) contacting the primary cells in the first well with the agent; (c) taking a first measurement of a property of the primary cells or media components in the first well; and (d) comparing the first measurement to a reference standard, wherein a difference between the first measurement and the reference standard indicates that the agent modifies the property. In certain embodiments, the reference standard is a predetermined value, a baseline measurement obtained from the first well before contacting the cells with the agent, or a negative control measurement taken from a second well comprising primary cells that have been contacted with a negative control. Some embodiments further comprise comparing the reference standard to a positive control measurement. In certain embodiments, the positive control measurement is generated by: (i) obtaining a second well comprising the primary cells in the presence of the cell culture medium; (ii) contacting the primary cells in the second well with the positive control; (iii) taking a second measurement of the property of the primary cells or the media components in the second well thereby generating the positive control measurement, wherein a difference between the positive control measurement and the reference standard indicates that a change in the property is detectable.

In certain embodiments, the property of the primary cells is selected from a list comprising: expression of one or more proteins, expression of one or more polynucleotides, activity of a secondary messenger system, ion transport, amino acid transport, peptide transport, protein transport, monosaccharide transport, lipid transport, bile acid transport, organic molecule transport, small molecule transport, acid/base transport, polysaccharide metabolism, protein metabolism, peptide metabolism, lipid metabolism, small molecule metabolism, protein trafficking, protein localization, secretion of a hormone, secretion of metabolites, secretion of a peptide, secretion of monosaccharides, secretion of ions, secretion of small molecules, secretion of lipids, secretion of acid/base, post translational modification, changes in cell type, changes in number of cell types, apoptosis, rate of cell division, or composition of cell types, transepithelial electrical resistance measurement. In certain embodiments, the measurements are taken by performing an assay selected from the list consisting of: fluorescence microscopy, fluorometric assay, chromogenic assay, chemiluminescent assay, ion chromatography, HPLC, mass spectrometry, RNA sequencing, DNA sequencing, ELISA, enzyme assays, flow cytometry, FACS, TUNEL assay, viability assay, proliferation assay, chelation assay, immunocytochemistry, western blot analysis, qPCR analysis, radiometric changes, microarray analysis, voltohm meter. In certain embodiments, the agent is an antibody, a natural or chemically modified polypeptide, a natural or chemically modified oligopeptide, a natural, unnatural, or chemically modified amino acid, a polynucleotide, a natural or chemically modified oligonucleotide, a natural or chemically modified mononucleotide, a lipopeptide, an antimicrobial, a small molecule, or a pharmaceutical molecule.

In certain embodiments, the primary cells originate from the small intestine. In certain embodiments, the primary cells originate from the colon. In certain embodiments, the negative or positive control agent is a control agent. In certain embodiments, the negative control is a vehicle control. In certain embodiments, the first and the second wells are standard wells. In certain embodiments, the first and the second wells are transwells. In certain embodiments, the transwells are on a multiple-well plate, selected from a 24-well plate, a 96-well plate, a 384-well plate, and a 1536-well plate. In certain embodiments, the property of the primary cells is ion transport. In certain embodiments, the ion is selected from one or more of a sodium ion, a chloride ion, a potassium ion, a phosphate ion, a bicarbonate ion, and any combination thereof. In certain embodiments, the property of the primary cells is secretion of a hormone. In certain embodiments, the hormone is an incretin. In certain embodiments, the incretin is a glucagon-like peptide. In certain embodiments, the agent is a TGR5 agonist.

Also included are methods of performing a high throughput screen, comprising performing a screen with a plurality of agents on a primary cell culture to identify agents within the plurality of agents that modifies a property of the primary cell culture, wherein the primary cell culture originates from intestine or colon, and wherein each agent of the plurality of agents is screened according to a methods described herein. In certain embodiments, the plurality of agents comprises at least 500 agents, at least 2,000 agents, or at least 10,000 agents.

Also included are methods of characterizing a site of action of an agent in a culture of primary cells generated according to any of the methods described herein, comprising: (a) obtaining a first well comprising the primary cells in the presence of a cell culture medium comprising EGF, a BMP inhibitor and optionally an LGR5 activator and a Wnt signaling agonist, wherein the primary cells originate from a small intestine or a colon, and wherein the primary cells comprise an apical membrane; (b) contacting the primary cells in the first well with the agent, wherein the agent contacts the primary cells at a site; (c) taking a first measurement of a property of the primary cells or media components in the first well; and (d) comparing the first measurement to a reference standard; wherein a difference between the first measurement and the reference standard indicates that the agent modifies the property at the site, and wherein no difference between the measurement and the reference indicates that the agent does not modify the property at the site. In certain embodiments, the reference standard is a predetermined value, a baseline measurement obtained from the first well before contacting the cells with the agent, or a negative control measurement taken from a second well comprising primary cells that have been contacted with a negative control. In certain embodiments, the site is selected from a membrane, cytosol, an apical membrane, an extracellular region of the apical membrane, a basolateral membrane, an extracellular region of the basolateral membrane, a protein, a ligand binding site of the protein, a membrane spanning protein, a cytosolic region of the membrane spanning protein, an extracellular region of the membrane spanning protein, an apical membrane spanning protein, a basolateral membrane spanning protein, a cytosolic region of the apical membrane spanning protein, an extracellular region of the apical membrane spanning protein, a cytosolic region of the basolateral membrane spanning protein, the extracellular region between cells, and an extracellular region of the basolateral membrane spanning protein. In certain embodiments, the property of the primary cells is selected from expression of one or more proteins, expression of one or more polynucleotides, activity of a secondary messenger system, ion transport, amino acid transport, peptide transport, protein transport, monosaccharide transport, lipid transport, bile acid transport, organic molecule transport, small molecule transport, acid/base transport, polysaccharide metabolism, protein metabolism, peptide metabolism, lipid metabolism, small molecule metabolism, protein trafficking, protein localization, secretion of a hormone, secretion of metabolites, secretion of a peptide, secretion of monosaccharides, secretion of ions, secretion of small molecules, secretion of lipids, secretion of acid/base, post translational modification, changes in cell type, changes in number of cell types, apoptosis, rate of cell division, transepithelial electrical resistance or composition of cell types. In certain embodiments, the measurement is taken by performing an assay selected from the list consisting of: fluorescence microscopy, fluorometric assay, chromogenic assay, chemiluminescent assay, ion chromatography, HPLC, mass spectrometry, RNA sequencing, DNA sequencing, ELISA, enzyme assays, flow cytometry, FACS, TUNEL assay, viability assay, proliferation assay, chelation assay, immunocytochemistry, western blot analysis, qPCR analysis, radiometric changes, microarray analysis, voltohm meter. In certain embodiments, the agent is an antibody, a natural or chemically modified polypeptide, a natural or chemically modified oligopeptide, a natural, unnatural, or chemically modified amino acid, a polynucleotide, a natural or chemically modified oligonucleotide, a natural or chemically modified mononucleotide, a lipopeptide, an antimicrobial, a small molecule, or a pharmaceutical molecule. In certain embodiments, the primary cells originate from the small intestine. In certain embodiments, the primary cells originate from the colon. In certain embodiments, the well is a standard well or a transwell.

Certain methods further comprise: (e) identifying the site of action where the agent contacts the primary cells. In certain embodiments, identifying the site of action comprises: (i) obtaining a third well comprising the primary cells in the presence of the cell culture medium; (ii) contacting the primary cells in the third well with an with the agent and with a competitor, wherein the competitor contacts the primary cells at a known site and wherein the competitor does not modify the property; (iii) taking a second measurement of the property of the primary cells or the media components in the third well; and (iv) comparing the second measurement to the first measurement and the reference standard, wherein if step (d) indicated that the agent modifies the property, then a difference between the third measurement and the reference standard and a similarity between the first and third measurement indicates that the agent does not modify the property at the known site; and wherein if step (d) indicated that the agent modifies the property, then a difference between the third measurement and the first measurement and a similarity between the first and third measurement indicates that the agent modifies the property at the known site. In certain embodiments, the competitor is an antibody, a natural or chemically modified polypeptide, a natural or chemically modified oligopeptide, a natural, unnatural, or chemically modified amino acid, a polynucleotide, a natural or chemically modified oligonucleotide, a natural or chemically modified mononucleotide, a lipopeptide, an antimicrobial, a small molecule, or a pharmaceutical molecule. In certain embodiments, the agent comprises a detectable label, and wherein step (e) comprises measuring the detectable label at the site, whereby the presence of the detectable label at the site indicates that the agent modifies the property at the site. In certain embodiments, the detectable label is a fluorophore or a radioactive isotope. In certain embodiments, the site of action is the cytosol, the apical membrane, the basolateral membrane, a cytosolic region of an apical membrane spanning protein, intracellular space between cells, an extracellular region of an apical membrane spanning protein. In certain embodiments, the agent is a DRA inhibitor, a TGR5 inhibitor, or a TGR5 agonist. In certain embodiments, the primary cells further comprise a basolateral membrane; and wherein the well is a transwell comprising a first compartment comprising the cell culture medium and a second compartment comprising the cell culture medium; wherein the cell culture medium of the first compartment contacts the apical membrane of the primary cells; wherein the cell culture medium of the second compartment contacts the basolateral membrane of the primary cells; and wherein the site is the apical membrane, the basolateral membrane, the extracellular region of the apical membrane, the extracellular region of the basolateral membrane, the cytosolic region of an apical membrane spanning protein, the extracellular region of an apical spanning protein, the cytosolic region of a basolateral membrane spanning protein, or the extracellular region of a basolateral membrane spanning protein.

In some embodiments, step (b) comprises: contacting the agent to the cell culture medium of the first compartment, thereby contacting the agent to the apical membrane; wherein a difference between the measurement and the reference standard indicates the extracellular region of the apical membrane as the site of action, and wherein no difference between the measurement and the reference standard indicates that the extracellular region of the apical membrane is not the site of action.

In some embodiments, step (b) comprises: contacting the agent to the cell culture medium of the second compartment, thereby contacting the agent to the basolateral membrane; wherein a difference between the measurement and the reference standard indicates the extracellular region of the basolateral membrane as the site of action, and wherein no difference between the measurement and the reference standard indicates that the extracellular region of the basolateral membrane is not the site of action.

Certain embodiments further comprise the steps of: (e) obtaining a second well comprising primary cells in the presence the cell culture medium wherein the primary cells originate from same source as the primary cells of step (a); (f) contacting the primary cells in the second well with the agent, wherein the agent contacts the primary cells at a site different from the site of step (b); (g) taking a second measurement of the property of the primary cells or the media components in the second well; and (h) comparing the first measurement to a reference standard, wherein a difference between the measurement of the primary cells in the first well and the reference standard and no difference between the measurement of the primary cells in the second well and the reference standard indicates that the site of action is the site contacted by the agent in the first well, wherein a difference between the measurement of the primary cells in the second well and the reference standard and no difference between the measurement of the primary cells in the first well and the reference standard indicates that the site of action is the site contacted by the agent in the second well.

In certain embodiments, step (b) comprises: contacting the agent to the cell culture medium of the first compartment, thereby contacting the agent to the apical membrane; and wherein step (f) comprises: contacting the agent to the cell culture medium of the second compartment, thereby contacting the agent to the basolateral membrane; and wherein a difference between the measurement of the primary cells in the first well and the reference standard and no difference between the measurement of the primary cells in the second well and the reference standard indicates that the site of action is the extracellular region of the apical membrane; and wherein a difference between the measurement of the primary cells in the second well and the reference standard and no difference between the measurement of the primary cells in the first well and the reference standard indicates that the site of action is the extracellular region of the basolateral membrane.

In certain embodiments, the property is GLP secretion. In certain embodiments, the agent is an ion transport antagonist and the property is ion transport. In certain embodiments, the ion is selected from one or more of a potassium ion, a chloride ion, and a sodium ion.

Also included are method for identifying or detecting at least one protein within a plurality of proteins present at a site of interest in primary cell culture generated by any of the methods described herein, comprising the steps of: (a) obtaining a well containing primary cells, wherein the primary cells originate from small intestine or colon; (b) isolating the plurality of proteins present at the site of interest; and (c) performing an analysis on the plurality of proteins, wherein the analysis comprises a technique that can detect or identify at least one protein; thereby detecting or identifying the at least one protein present at the site of interest; wherein the well is a transwell comprising a first compartment and a second compartment, and wherein the first compartment comprises a cell culture medium that contacts an extracellular surface of an apical membrane of the primary cells, and wherein the second compartment comprises a cell culture medium that contacts an extracellular surface of a basal membrane of the primary cells; and wherein identifying or detecting the at least one protein comprises quantifying an amount of the at least one protein present in the primary cell culture. In certain embodiments, the well is a transwell and wherein the primary cells are differentiated.

In certain embodiments, isolating the plurality of proteins present at the site of interest comprises: (i) contacting the primary cells with a tag molecule, wherein the tag molecule binds to or attaches to the plurality of proteins or binds to or attaches to the site of interest comprising the plurality of proteins; and (ii) removing the tag molecule from the primary cell culture, and thereby removing the plurality of proteins bound to the tag molecule or the site of interest bound to the tag molecule. In certain embodiments, the tag molecule binds to or attaches the plurality of proteins at the site of interest, wherein the site of interest is the extracellular surface of the apical membrane of the primary cells, and wherein step (i) comprises: contacting the tag molecule to the cell culture medium in the first compartment of the transwell, thereby contacting the tag molecule to an extracellular surface of the apical membrane of the primary cells. In certain embodiments, the tag molecule binds to or attaches to the plurality of proteins at the site of interest, wherein the site of interest is the extracellular surface of the basal membrane of the primary cells, and wherein step (i) comprises: contacting the tag molecule to the cell culture medium in the second compartment of the transwell, thereby contacting the tag molecule to an extracellular surface of the basal membrane of the primary cells.

In certain embodiments, step (ii) comprises:—homogenizing the primary cell culture; —contacting a bead to the homogenized primary cell culture, wherein the bead binds to the tag molecule; and—removing the beads from the homogenized primary cell culture; thereby removing the tag molecule from the primary cell culture.

In certain embodiments, the tag molecule is a biotin molecule, and wherein the bead is coated with avidin molecules. In certain embodiments, the biotin molecule is sulfo-NHS-SS biotin, and/or wherein the avidin molecule is NeutrAvidin. In certain embodiments, the site of interest is a subpopulation of cells within the primary cell culture, and wherein isolating the plurality of proteins present at the site of interest comprises: (i) fluorescently labeling the subpopulation of cells within the primary cell culture; (ii) dissociating cells of the primary cell culture; (iii) separating dissociated cells that are fluorescently labeled from cells not that are not fluorescently labeled with fluorescence-activated cell sorting (FACS); and (iv) homogenizing the cells that are fluorescently labeled; thereby generating a homogenized sample comprising a plurality of proteins from the site of interest.

In certain embodiments, step (i) comprises contacting the primary cell culture with an antibody conjugated to a fluorescent label, wherein the antibody conjugated to a fluorescent label preferentially binds to the subpopulation of cells within the primary cell culture.

In certain embodiments, the antibody conjugated to a fluorescent label binds to a cell surface protein expressed by the subpopulation of cells; wherein the cell surface protein is not expressed on cells in the primary cell culture that are not in the subpopulation.

In certain embodiments, step (i) comprises inducing expression of a fluorescent protein in the subpopulation of cells within the primary cell culture. In certain embodiments, expression of the fluorescent protein in the subpopulation of cells is induced by delivering a nucleotide encoding the fluorescent protein. In certain embodiments, delivering the nucleotide comprises transfecting the subpopulation of cells with a virus. In certain embodiments, the primary cell culture is originated from a transgenic mouse, and wherein the fluorescent protein is a transgene, and wherein expression of the transgene is induced in the subpopulation of cells within the primary cell culture, and wherein the transgene is not induced in cells within the primary cell culture that are not in the subpopulation. In certain embodiments, the subpopulation of cells within the primary cell culture is a cell type, and wherein the cell type is an absorptive enterocytes, a goblet cell, an enteroendocrine cell, a Paneth cell, transit amplifying cells, or stem cell. In certain embodiments, wherein the site of interest is a subcellular fraction of cells of the primary cell culture, and wherein isolating the plurality of proteins present at the site of interest further comprises: (i) homogenizing the cells of the primary cell culture, and (ii) isolating the subcellular fraction of the homogenized sample with a subcellular fractionation technique.

In certain embodiments, the site of interest is cytosol, plasma membrane, nuclei, mitochondria, or ribosome. In certain embodiments, the method comprises identifying multiple proteins within a plurality of proteins present at the site of interest, and wherein performing an analysis on the plurality of proteins comprises: (i) purifying the plurality of proteins; and (ii) identifying the plurality of proteins with mass spectrometry, a protein micro array, or western blot analysis.

Also included are methods for determining if expression at a site of interest of at least one protein within a first plurality of proteins present at the site of interest in a primary cell culture is altered by an agent comprising: (a) obtaining a first well containing the primary cells, wherein the primary cells originate from small intestine or colon; (b) contacting an agent to the primary cell culture in the first well; (c) isolating the first plurality of proteins present at the site of interest; (c) performing an analysis on the plurality of proteins, wherein the analysis comprises a technique that can detect or identify at least one protein; and (d) comparing the results of the analysis to a reference standard, thereby detecting or identifying at least one protein present at a site of interest; wherein the primary cell culture is generated by the methods described herein; wherein the well is a transwell comprising a first compartment and a second compartment, and wherein the first compartment comprises a cell culture medium that contacts an extracellular surface of an apical membrane of the primary cells, and wherein the second compartment comprises a cell culture medium that contacts an extracellular surface of a basal membrane of the primary cells; and wherein a difference between the results of the analysis of the plurality of proteins from the first well and the reference standard indicates that the agent modifies expression of the at least one protein at the site of interest. In certain embodiments, the reference standard is predetermined. In certain embodiments, comparing the results of the analysis to a reference standard comprises the steps of: (i) obtaining a second well comprising the primary cells in the presence of the cell culture medium; (ii) contacting the primary cells in the second well with a negative control; (iii) isolating the plurality of proteins present at the site of interest; (iv) performing a second analysis on the plurality of proteins, wherein the analysis comprises a technique that can detect or identify at least one protein; and (v) comparing the results of the first analysis to results of second measurement. Some embodiments comprise: (e) comparing the results of the first analysis and the second analysis to a positive control, wherein a difference between the results of the reference standard and the positive control and the reference standard indicates that a modification of the expression of the at least one protein at a site of interest is detectable.

In certain embodiments, step (e) comprises: (e)(i) obtaining a third well comprising the primary cells in the presence of the cell culture medium; (e)(ii) contacting the primary cells in the third well with the positive control agent; (e)(iii) isolating the plurality of proteins present at the site of interest in the primary cells of the third well; (e)(iv) performing a third analysis on the plurality of proteins present at the site of interest in the primary cells of the third well, wherein the analysis comprises a technique that can detect or identify at the least one protein; and (e)(v) comparing the results of the first analysis to results of second analysis and third analysis, thereby comparing the first measurement to the negative and the positive control.

In certain embodiments, contacting the agent to the primary cell culture in the first well and optionally the negative control to the primary cell culture in the second well and optionally the positive control to the primary cell culture in the third well comprises the steps of: contacting the primary cells with a tag molecule, wherein the tag molecule binds to or attaches to proteins within the plurality of proteins or binds to or attaches to the site of interest comprising the plurality of proteins; and removing the tag molecule from the cell culture, and thereby removing the proteins bound to the tag molecule or the site of interest bound to the tag molecule; thereby isolating the plurality of proteins present at the site of interest. In certain embodiments, tag molecule binds to or attaches the plurality of proteins at the site of interest, wherein the site of interest is the extracellular surface of the apical membrane of the primary cells, and wherein contacting the primary cells with a tag molecule comprises: contacting the tag molecule to the cell culture medium in the first compartment of the transwell, thereby contacting the tag molecule to an extracellular surface of an apical membrane of the primary cells. In certain embodiments, the tag molecule binds to or attaches the plurality of proteins at the site of interest, wherein the site of interest is the extracellular surface of the basal membrane of the primary cells, and wherein contacting the primary cells with a tag molecule comprises: contacting the tag molecule to the cell culture medium in the second compartment of the transwell, thereby contacting the tag molecule to an extracellular surface of a basal membrane of the primary cells. In certain embodiments, removing the tag molecule from the cell culture comprises the steps of: homogenizing the primary cell culture; contacting a bead to the homogenized primary cell culture, wherein the bead binds to the tag molecule; and removing the beads from the homogenized primary cell culture, thereby removing the tag molecule from the primary cell culture. In certain embodiments, the tag molecule is a biotin molecule, and wherein the bead is coated with avidin molecules. In certain embodiments, the biotin molecule is sulfo-NHS-SS biotin and/or wherein the avidin molecule is NeutrAvidin. In certain embodiments, the site of interest is a subpopulation of cells within the primary cell culture, and wherein isolating the first plurality of proteins present at the site of interest comprises the steps of: fluorescently labeling the subpopulation of cells within the primary cell culture; dissociating cells of the primary cell culture; separating dissociated cells that are fluorescently labeled from cells not that are not fluorescently labeled with fluorescence-activated cell sorting (FACS); and homogenizing the cells that are fluorescently labeled; thereby generating a homogenized sample comprising a plurality of proteins from the site of interest. In certain embodiments, the fluorescently labeling the subpopulation of cells within the primary cell culture comprises contacting the primary cell culture with an antibody conjugated to a fluorescent label, wherein the antibody conjugated to a fluorescent label preferentially binds to the subpopulation of cells within the primary cell culture. In certain embodiments, the antibody conjugated to a fluorescent label binds to a cell surface protein expressed by the subpopulation of cells; wherein the cell surface protein is not expressed on cells in the primary cell culture that are not in the subpopulation. In certain embodiments, fluorescently labeling the subpopulation of cells within the primary cell culture comprises inducing expression of a fluorescent protein in the subpopulation of cells within the primary cell culture. In certain embodiments, the expression of the fluorescent protein in the subpopulation of cells is induced by delivering a nucleotide encoding the fluorescent protein. In certain embodiments, delivering the nucleotide comprises transfecting the subpopulation of cells with a virus.

In certain embodiments, the primary cell culture is originated from a transgenic mouse, and wherein the fluorescent protein is a transgene, and wherein expression of the transgene is induced in the subpopulation of cells within the primary cell culture, and wherein the transgene is not induced in cells within the primary cell culture that are not in the subpopulation. In certain embodiments, the subpopulation of cells within the primary cell culture is a cell type, and wherein the cell type is absorptive enterocytes, a goblet cell, an enteroendocrine cell, a Paneth cell, a transit amplifying cell, or a stem cell.

In certain embodiments, the site of interest is a subcellular fraction of cells of the primary cell culture, and wherein isolating the first plurality of proteins present at the site of interest of the primary cell culture in the first well, and optionally isolating the second plurality of proteins present in the primary cell culture of the second well, and optionally isolating the third plurality present in the primary cell culture in the third well comprises: homogenizing the cells of the primary cell culture; and isolating the subcellular fraction of the homogenized sample with a subcellular fractionation technique.

In certain embodiments, the site of interest is cytosol, plasma membrane, nuclei, mitochondria, or ribosome. In certain embodiments, the method comprises identifying multiple proteins within a plurality of proteins present at the site of interest, and wherein performing an analysis on the plurality of proteins comprises: purifying the plurality of proteins; and identifying the plurality of proteins with mass spectrometry, a protein micro array, or western blot analysis.

Also included are methods of determining the localization of at least one protein of interest in a primary cell culture derived from small intestine or colon comprising: (a) obtaining a first well of primary cultured cells; (b) contacting the at least one protein with a detectable label; and (c) visualizing the detectable label with a microscope, thereby determining the localization of the at least one protein of interest; wherein the primary cells are generated by any of the methods described herein; wherein the first well is a transwell comprising a first compartment and a second compartment, and wherein the first compartment comprises a cell culture medium that contacts an extracellular surface of an apical membrane of the primary cells, and wherein the second compartment comprises a cell culture medium that contacts an extracellular surface of a basal membrane of the primary cells. In certain embodiments, the primary cell culture in the first well is contacted with an agent. In certain embodiments, the method comprises: (d) obtaining a second well of primary cultured cells; (e) contacting the at least one protein with the detectable label; (f) visualizing the detectable label with a microscope; and (g) comparing the results of step (c) with step (f), wherein the second well is not contacted with an agent or is contacted with a negative control agent; and wherein the first well is a transwell comprising a first compartment and a second compartment, and wherein the first compartment comprises a cell culture medium that contacts an extracellular surface of an apical membrane of the primary cells, and wherein the second compartment comprises a cell culture medium that contacts an extracellular surface of a basal membrane of the primary cells; thereby determining if the agent alters the localization of the protein of interest.

In certain embodiments, the primary cell culture second is not contacted with an agent. In certain embodiments, the second well is contacted with a negative control agent. In certain embodiments, the method comprises the steps of: (h) obtaining a third well of primary cultured cells; (i) contacting the at least one protein with the detectable label; (j) visualizing the detectable label with a microscope; and (k) comparing the results of step (c) with step (f) and step (j); wherein the third well is a transwell comprising a first compartment and a second compartment, and wherein the first compartment comprises a cell culture medium that contacts an extracellular surface of an apical membrane of the primary cells, and wherein the second compartment comprises a cell culture medium that contacts an extracellular surface of a basal membrane of the primary cells; thereby determining if the agent alters the localization of the protein of interest. In certain embodiments, contacting the at least one protein with the detectable label comprises the steps of: contacting a primary antibody that specifically binds to the at least one protein of interest to the primary cell culture: and contacting the primary antibody with a detectable label. In certain embodiments, contacting the primary antibody with a detectable label comprises conjugating the detectable label to the primary antibody prior to contacting the primary antibody to the cell culture. In certain embodiments, contacting the primary antibody with a detectable label comprises contacting a secondary antibody conjugated with a detectable label to the primary antibody. In certain embodiments, the detectable label is attached at the C terminal or the N terminal of the at least one protein of interest, wherein the at least one protein of interest and the detectable label are expressed from a transgene; and wherein the primary cell culture is transfected with the transgene. In certain embodiments, the detectable label is a fluorescent label.

### BRIEF DESCRIPTION OF THE FIGURES

FIG. 1A-FIG. 1G illustrate characterization of mouse colonic monolayer cultures including: transepithelial electrical resistance (TEER) values of proximal and distal colonic cultures (FIG. 1A); immunostaining for major cell lineages of the colon and confocal microscopy z-stack imaging of apical DRA (Slc26a3) localization (FIG. 1B); absorption of water from the apical compartment to the basolateral compartment in transwell-cultured distal colonic cultures and apical secretion of acid in proximal colonic cultures (FIG. 1C); ion chromatography analysis of media in the apical and basolateral compartments of proximal and distal colonic cultures (FIG. 1D); RNA expression patterns in proximal and distal colonic cultures and tissue (FIG. 1E); gene expression patterns of differentiated and un-differentiated distal colonic cultures (FIG. 1F); and a heat map comparing gene expression in cultures versus proximal colonic tissue and distal colonic tissue (FIG. 1G). AP=apical; BL=basolateral; DAPI=4′,6-diamidino-2-phenylindole.

FIG. 2A-FIG. 2H illustrate characterization of mouse small intestinal monolayer cultures including: a Pearson correlation coefficient (PCC) plot for global gene expression in jejunal cultures vs. jejunum villus tissue against TEER for jejunal cultures grown under various conditions (FIG. 2A); PCC for global gene expression in jejunal cultures versus jejunal villus or crypt tissue differentiated with varying concentrations of BMP2, BMP4, or BMP7 (FIG. 2B); PCC for global gene expression in duodenal, jejunal, and ileal cultures versus villus tissue for different intestinal segments (FIG. 2C); a heat map comparing gene expression in cultures versus tissue for duodenum, jejunum, and ileum (FIG. 2D); immunostaining for major cell lineages in jejunal cultures (FIG. 2E); pH analysis of media in the apical and basolateral compartments for cultures from duodenum, jejunum, and ileum (FIG. 2F); Ion concentrations in the apical and basolateral compartments of jejunal and ileal cultures (FIG. 2G); total ion amount in the apical compartment of mouse jejunal and ileal cultures on day 7 and day 5 (FIG. 2H). ANPEP=alanine aminopeptidase; AP=apical; BL=basolateral; BMP=bone morphogenetic protein; E=epidermal growth factor; T=thiazovivin.

FIG. 3A-FIG. 3F illustrate characterization of human intestinal monolayer cultures including: sodium, potassium, and chloride ion transport by differentiated human distal colonic cultures (FIG. 3A); immunostaining for major cell lineages in distal colonic cultures (FIG. 3B); comparison of ion-transporter expression in cultured cells and tissue from the proximal and distal colon (FIG. 3C); Pearson correlation coefficients for global gene expression profiles from patient duodenal tissue samples and patient-derived cultures grown under different seeding/differentiation media conditions (FIG. 3D); immunostaining for major cell lineages of ileal cultures (FIG. 3E); and a heat map comparing gene expression in cultures versus tissue for duodenum, ileum, and distal colon (FIG. 3F). A=A83-01; E=epidermal growth factor; N=noggin; Nic=nicotinamide; R=R-spondin 1; S=SB202190; T=thiazovivin; W=Wnt3a; Y=Y-27632.

FIG. 4 illustrates results of a screen of approximately 2,000 compounds for inhibition of K⁺ transport from the apical to the basolateral compartments of mouse distal colonic cultures.

FIG. 5 shows Pearson correlation coefficients for global gene expression of mouse ileal cultures differentiated with varying concentrations of BMP2, BMP4, or BMP7 versus villi or crypts from the duodenum, jejunum, and ileum.

FIG. 6 shows PCCs for global gene expression of human duodenal cultures grown under different seeding/differentiation media conditions (patients 8 and 9) versus tissue samples (patients 11 and 13).

FIG. 7 shows validation of the PCC as a quantitative measurement of gene expression similarity between cultured cells and villus tissue.

FIG. 8 illustrates water absorption and movement of ions in human distal colonic cultures grown in the presence or absence of A83-01. A=A83-01; E=epidermal growth factor; N=noggin; R=R-spondin 1; W=Wnt3a.

FIG. 9A-FIG. 9B illustrate a comparison of TEER values (FIG. 9A) and global RNA profiles (FIG. 9B) in cultured human distal colonic cells seeded/differentiated in different media. A=A83-01; D=day; E=epidermal growth factor; N=noggin; R=R-spondin 1; T=thiazovivin; Nic=nicotinamide; W=Wnt3a; Y=Y-27632.

FIG. 10 illustrates the expression levels of cell lineage marker genes associated with goblet, enteroendocrine and absorptive cell types from human distal colonic cultures seeded and differentiated in different media. A=A83-01; D=day; E=epidermal growth factor; N=noggin; R=R-spondin 1; S=SB202190; T=thiazovivin; Nic=nicotinamide; W=Wnt3a; Y=Y-27632.

FIG. 11 shows goblet cell staining and TEER of human duodenal cultures prepared using different seeding/differentiation media combinations. A=A83-01; E=epidermal growth factor; N=noggin; R=R-spondin 1; S=SB202190; T=thiazovivin; TEER=transepithelial electrical resistance; Nic=nicotinamide; W=Wnt3a; Y=Y-27632.

### DETAILED DESCRIPTION

The present disclosure provides methods for generating intestinal monolayer cultures from primary human and murine intestinal samples. These methods can be used to generate cultures for use in a high-throughput system for compound evaluation and drug discovery platforms.

### A. Definitions

As used in this specification and the appended claims, the singular forms “a,” “an,” and “the” include plural references unless the content clearly dictates otherwise. As used in this specification, the term “and/or” is used in this disclosure to either “and” or “or” unless indicated otherwise.

As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

“Isolated” refers to a material that is free to varying degrees from components which normally accompany it as found in its native state. “Isolate” denotes a degree of separation from original source or surroundings.

As used herein, an “agent” refers to a compound or molecule capable of exerting a particular physiological effect on a cell. Agents can include, but are not limited to, small molecules, proteins, antibodies or antigen-binding fragments thereof, and/or nucleic acids. In some embodiments, an agent encompasses compounds or molecules added to one or more media used in the culture of murine and/or human intestinal cells. In some embodiments, an agent encompasses compounds or molecules

As used herein, an “agonist” is an agent that causes an increase in the activity of a signaling pathway (e.g., an agent that increases the activity of the Wnt signaling pathway such as Wnt3a). Herein, reference to an agent as an agonist is based on the effect of the agent on a pathway as a whole, and not necessarily its effect on a specific pathway component. For example, an agonist may be an activator (e.g., may increase the expression and/or activity of a particular receptor, gene, and/or protein, for example Wnt3a) or an inhibitor (e.g., may decrease the expression and/or activity of a particular receptor, gene, and/or protein, for example a GSK3 inhibitor), so long as the overall effect is an increase in the activity of a particular pathway as a whole. For example, an agonist may directly increase the activity of a particular signaling pathway through binding to its cognate receptor and activating signaling pathways immediately downstream of that receptor. Alternatively, an agonist may indirectly increase the activity of a particular signaling pathway, such as through the inhibition of negative regulator of a pathway.

An “antagonist” is an agent that causes a decrease in the activity of a signaling pathway (e.g., an agent that decreases the activity of TGFβ signaling). Herein, reference to an agent as an antagonist is based on the effect of the agent on a pathway as a whole, and not necessarily its effect on a specific pathway component. As such, an antagonist may be an activator (e.g., may increase the expression and/or activity of a particular receptor, gene and/or protein) or an inhibitor (e.g., may decrease the expression and/or activity of a particular receptor, gene, and/or protein), so long as the overall effect is a decrease in the activity of a particular pathway as a whole. In some embodiments, the actions of an antagonist may be direct, such as through physically preventing an interaction between a ligand/receptor pair (e.g., a blocking antibody) and/or through increasing the activity of inhibitory signaling pathways immediately downstream of its cognate receptor. In some embodiments, the actions of an antagonist may be indirect, such as through the activation and/or maintained expression of a pathway inhibitor.

An “activator” as used herein refers to an agent that causes an increase in the activity or expression of a target receptor, gene, and/or protein.

An “inhibitor” refers to an agent that causes a decrease in the expression or activity of a target receptor, gene, and/or protein.

The term “sample” refers to a volume and/or mass obtained, provided, and/or subjected to analysis. In some embodiments, a sample comprises a tissue sample, cell sample, a fluid sample, and the like. In some embodiments, a sample is taken from a subject (e.g., a human or animal subject). In some embodiments, a tissue sample comprises a portion of tissue taken from an intestine.

In some embodiments, a “sample” is a “primary sample” in that it is obtained directly from a source (e.g., a subject). In some embodiments, a primary sample is a “primary cell,” wherein a cell is obtained directly from a subject. In some embodiments, a primary sample encompasses the processing of a primary sample, for example culturing a primary sample to expand and/or differentiate one or more cells comprised within the sample. In some embodiments, primary cells and/or primary samples are obtained from a mammalian subject. In further embodiments, primary cells and/or primary samples are obtained from a human or a murine subject.

“Population” of cells refers to any number of cells greater than 1. For example a population of cells is at least two, at least 1×10³ cells, at least 1×10⁴ cells, at least at least 1×10⁵ cells, at least 1×10⁶ cells, at least 1×10⁷ cells, at least 1×10⁸ cells, at least 1×10⁹ cells, at least 1×10¹⁰ cells, or more cells. A population of cells may refer to an in vitro population (e.g., a population of cells in culture) or an in vivo population (e.g., a population of cells residing in a particular tissue).

“Organoid” refers to a cell cluster or aggregate that resembles an organ, or part of an organ, and possesses cell types relevant to that particular organ.

The terms “culture,” “cell culture,” and “tissue culture” are used interchangeably herein and refer to a system capable of maintaining, facilitating, and/or enhancing the growth, function, and/or viability of a cell or a tissue. A cell culture system comprises multiple factors and conditions including cell or tissue type, basal media, media additives and supplements, feed schedule, passage schedule, culture temperature, temperature shifts, humidity, degree of aeration, pH, seeding cell density, CO₂ level, oxygen level, the type of solid support (e.g., a well or insert of a culture plate or a culture flask). In some embodiments, the cell culture system is three-dimensional (3D). In some embodiments, the cell culture system is two-dimensional (2D). In some embodiments, the cell culture system is a combination of 2D and 3D culture conditions (e.g., cells are first cultured in a 3D culture system and then passaged into a 2D culture system).

“Media” and “cell culture media” are used interchangeably herein and refer to a solution, solid, or semi-solid (e.g., Matrigel) capable of supporting cell growth and/or differentiation. “Basal media” refers to media that has not been modified with additives or supplements. “Supplemented media” refers to a basal media (e.g., basal media) that has been modified by the addition of one or more additives or supplements. Herein, the terms “additive” or “supplement” may refer to any compound, molecule, or agent that is added to a basal media. Additives and supplements may include, but are not limited to, serum (e.g., fetal bovine serum and fetal calf serum), amino acids (e.g., L-glutamine), chelating agents (e.g., EDTA), growth factors, antibiotics, antioxidants (e.g., N-acetylcysteine), vitamins, agonists, antagonists, activators, and/or inhibitors.

### B. Media and Cell Culture Conditions

In some embodiments, the present disclosure provides methods for culturing primary intestinal cells in a monolayer comprising isolating cells from a mammalian tissue sample, plating the cells in a seeding medium, growing the cells in a growth medium and differentiating the cells in a differentiation medium. Any of the culture conditions described herein (e.g., media, media components, type of solid supports, length of culture time, etc.) may be optimized to facilitate, promote, and/or maintain the growth and/or differentiation of a particular cell type derived from a particular source. In some embodiments, the methods described herein comprise culture conditions that have been optimized to promote the growth and/or differentiation of a cell derived from a particular intestinal segment of a human or a mouse intestine.

In some embodiments, cells are isolated from a tissue obtained from a mammalian subject (e.g., a human or a mouse). Typically, tissue samples are incubated for a pre-determined period of time in a dissociation medium or solution comprising one or more agents that facilitate the dissociation of cells from the structural components of a tissue. In some embodiments, the dissociation medium comprises a protease such a cysteine, serine, threonine, aspartic, glutamic, or metalloprotease. In some embodiments, the dissociation medium comprises a protease that cleaves extracellular matrix proteins (e.g., collagen, elastin, and/or fibrinectin) such as an elastase, a collagenase, and/or dispase. In some embodiments, the dissociation medium comprises dispase. The nature of the dissociation medium will depend on the type and source of the tissue. In some embodiments, the tissue is an intestinal tissue (e.g., a small or large intestine tissue) and is derived from a murine subject. In some embodiments, the tissue is an intestinal tissue (e.g., a small or large intestine tissue) and is derived from a human subject. In some embodiments, incubation of a tissue in a dissociation medium results in a heterogeneous cell suspension. In some embodiments, the heterogeneous cell suspension comprises one or more of enterocytes, goblet cells, enteroendocrine cells, Paneth cells, transit amplifying cells, and/or stem cells.

“Seeding medium,” as used herein refers to the medium used to resuspend the cell after isolation for plating the cell on a solid support (e.g., a culture plate or flask). For example, in some embodiments, cells are resuspended in a seeding medium after all isolation steps are complete and the resultant cell suspension is added to a culture plate or flask cells. In some embodiments, the seeding medium is a supplemented medium comprising one or more growth factors, inhibitors, activators, antagonists, and/or agonists.

The components of the seeding media may be modified based on the type and source of the tissue and the nature of the cell culture system. In some embodiments, the seeding media is optimized for seeding of primary cells in a 2D culture system. In some embodiments, the seeding medium is optimized for cells derived from the murine colon and comprises one or more of epidermal growth factor (EGF), a bone morphogenic protein (BMP) inhibitor, a leucine-rich repeat-containing G-protein coupled receptor (LGR5) activator, a Wnt signaling agonist, and a Rho-associated protein kinase (ROCK) inhibitor. In some embodiments, the seeding medium is optimized for cells derived from the murine colon and comprises EGF, Noggin, R-spondin-1, Wnt3a, and Y27632. In some embodiments, the seeding medium is optimized for cells derived from the murine small intestine and comprises EGF, a BMP inhibitor, an LGR5 activator, a Wnt signaling agonist, and a ROCK inhibitor. In some embodiments, the seeding medium is optimized for cells derived from the murine small intestine and comprises EGF, Noggin, R-spondin-1, Wnt3a, and Y27632. In some embodiments, the seeding medium is optimized for cells derived from a human colonic organoid and comprises EGF, a BMP inhibitor, an LGR5 activator, a Wnt signaling agonist, a TGFβ signaling antagonist, and a ROCK inhibitor. In some embodiments, the seeding medium is optimized for cells derived from a human colonic organoid and comprises EGF, Noggin, R-spondin-1, Wnt3a, A83-01, and thiazovivin. In some embodiments, the seeding medium is optimized for cells derived from a human small intestine organoid and comprises EGF, a BMP inhibitor, an LGR5 activator, a Wnt signaling agonist, a TGFβ signaling antagonist, and a ROCK inhibitor. In some embodiments, the seeding medium is optimized for cells derived from a human small intestine organoid and comprises EGF, Noggin, R-spondin-1, Wnt3a, A83-01, and Y27632.

In some embodiments, the seeding medium is modified in order to optimize cell adherence to a solid support to promote the generation of a two-dimensional monolayer cell culture of primary cells. In some embodiments, the solid support is a cell culture flask. In some embodiments, the solid support is a cell culture plate (e.g., a 6, 12, 24, 96, 384, or 1536-well plate). In some embodiments, the solid support is a cell culture plate with a transwell insert in one or more of the wells. In some embodiments, the solid support is a cell culture plate with a transwell insert in each of the wells (e.g., a 6, 12, 24, 96, 384, or 1536-well plate with a transwell insert in each well). In some embodiments, the solid support is coated with one or more extracellular matrix proteins. In some embodiments, the solid support is coated with a solution comprising 0.1 mg/mL-5 mg/mL extracellular matrix proteins. In some embodiments, the solid support is coated with a solution comprising 0.1 mg/mL-2 mg/mL extracellular matrix proteins. In some embodiments, the solid support is coated with a solution comprising 0.4 mg/mL extracellular matrix proteins.

“Growth medium” as used herein refers to a medium that facilitates the growth (e.g., proliferation and/or expansion) of a cell. For example, in some embodiments, cells are plated in a seeding medium and incubated for a predetermined amount of time, after which the seeding medium is removed and a growth medium is added to the cell culture. In some embodiments, the seeding media is removed from the culture and growth media is added 1 day after plating the cells. In some embodiments, the seeding media is removed from the culture and growth media is added 2, 3, 4, 5, 6, 7, 8, 9, 10 or more days after plating the cells. In some embodiments, the growth medium is a supplemented medium comprising one or more growth factors, inhibitors, activators, antagonists, and/or agonists.

The components of the growth media may be modified based on the type and source of the tissue and the nature of the cell culture system. In some embodiments, the growth media is optimized for seeding of primary cells in a 2D culture system. In some embodiments, the growth medium is optimized for cells derived from the murine colon and comprises one or more of EGF, a BMP inhibitor, an LGR5 activator, and a Wnt signaling agonist. In some embodiments, the growth medium is optimized for cells derived from the murine colon and comprises EGF, Noggin, R-spondin 1, and Wnt3a. In some embodiments, the growth medium is optimized for cells derived from the murine small intestine and comprises EGF, a BMP inhibitor, an LGR5 activator, a Wnt signaling agonist, and a ROCK inhibitor. In some embodiments, the growth medium is optimized for cells derived from the murine small intestine and comprises EGF, Noggin, R-spondin 1, Wnt3a, and Y27632. In some embodiments, the growth medium is optimized for cells derived from a human colonic organoid and comprises EGF, a BMP inhibitor, an LGR5 activator, a Wnt signaling agonist, a ROCK inhibitor, and a TGFβ signaling antagonist. In some embodiments, the growth medium is optimized for cells derived from a human colonic organoid and comprises EGF, Noggin, R-spondin 1, Wnt3a, thiazovivin, and A83-01. In some embodiments, the growth medium is optimized for cells derived from a human small intestine organoid and comprises EGF, a BMP inhibitor, an LGR5 activator, a Wnt signaling agonist, a ROCK inhibitor, and a TGFβ signaling antagonist. In particular embodiments, the growth medium is optimized for cells derived from a human small intestine organoid and comprises EGF, Noggin, R-spondin 1, Wnt3a, Y-27632, and A83-01.

In some embodiments, the cells are incubated in a growth media until the cells have reached a desired confluency. For example, in some embodiments, cells are incubated in a growth media until the cells have reach at least 50%, 60%, 70%, 80%, 90%, or 100% confluency. In some embodiments, the cells are incubated in a growth media until the culture demonstrates a predetermined transepithelial electrical resistance (TEER) value. For example, in some embodiments, the cells are incubated in a growth media until the culture demonstrates a TEER value of about 100 Ω·cm² or greater. In some embodiments, the TEER value is about 200, 300, 400, 500, 600, 700, 800, 900, 1000, 1100, 1200, 1300, 1400, 1500 Ω·cm² or greater. In some embodiments, the growth media is changed or replaced at regular intervals of time. For example, in some embodiments, the growth media is changed daily. In some embodiments, the growth media is changed every other day. In some embodiments, the growth media is changed every third day.

“Differentiation medium” as used herein refers to a medium that facilitates the differentiation of one or more cells in a cell culture system into a specialized or more a mature cell type. For example, in some embodiments, cells are plated in a seeding medium and incubated for a predetermined amount of time, after which the seeding medium is removed and a growth medium is added to the cell culture. In this illustrative example, after a predetermined amount of time or after the cells have demonstrated a desired characteristic (e.g., confluency or TEER) the growth media is removed and a differentiation media is added. In some embodiments, the growth media is removed from the culture and differentiation media is added at least two days after the growth media was initially added to the culture. In some embodiments, the growth media is removed from the culture and differentiation media is added at least 3, 4, 5, 6, 7, 8, 9, 10, or more days after growth media was initially added. In some embodiments, the growth media is removed from the culture and differentiation media is added when the cells have reached at least 50% confluency. In some embodiments, the growth media is removed from the culture and differentiation media is added when the cells have reached at least 60%, 70%, 80%, 90% or greater confluency. In some embodiments, the growth media is removed from the culture and differentiation media is added when the cells are 100% confluent. In some embodiments, the growth media is removed from the culture and differentiation media is added when the cells have a TEER value of at least 100 Ω·cm² or greater. In some embodiments, the growth media is removed from the culture and differentiation media is added when the cells have a TEER value of at least 150 Ω·cm² or greater. In some embodiments, the growth media is removed from the culture and differentiation media is added when the cells have a TEER value of at least 250 Ω·cm² or greater. In some embodiments, the growth media is removed from the culture and differentiation media is added when the cells have a TEER value of at least 100, 200, 300, 400, 500, 600, 700, 800, 900, 1000, 1100, 1200, 1300, 1400, 1500 Ω·cm² or greater. In some embodiments, the differentiation medium is a supplemented medium comprising one or more growth factors, inhibitors, activators, antagonists, and/or agonists.

The components of the differentiation media may be modified based on the type and source of the tissue and the nature of the cell culture system. In some embodiments, the differentiation media is optimized for differentiating primary cells in a 2D culture system. In some embodiments, the differentiation medium is optimized for cells derived from the murine colon and comprises one or more of EGF and a BMP inhibitor. In some embodiments, the differentiation media is optimized for cells derived from the murine colon and comprises EGF and Noggin. In some embodiments, the differentiation medium is optimized for cells derived from the murine small intestine and comprises one or more of EGF, a ROCK inhibitor, and a differentiation promoting factor. In such embodiments, the differentiation promoting factor may comprise a bone morphogenic protein (BMP) such as BMP2, BMP4, and/or BMP7. In some embodiments, the differentiation medium is optimized for cells derived from the murine small intestine and comprises EGF, thiazovivin, and BMP4. In some embodiments, the differentiation medium is optimized for cells derived from the murine small intestine and comprises EGF, thiazovivin, and BMP4. In some embodiments, the differentiation medium is optimized for cells derived from a human colon organoid and comprises one or more of EGF, a BMP inhibitor, an Lgr5 activator, and a TFGβ signaling antagonist. In some embodiments, the differentiation medium is optimized for cells derived from a human colon organoid and comprises EGF, Noggin, R-spondin 1, and A83-01. In some embodiments, the differentiation medium is optimized for cells derived from a human small intestine organoid and comprises one or more of EGF, a BMP inhibitor, and a TGFβ signaling antagonist. In some embodiments, the differentiation medium is optimized for cells derived from a human small intestine organoid and comprises EGF, Noggin, and A83-01.

In some embodiments, the cells are incubated in a differentiation media for a predetermined amount of time to allow for the cells to differentiate into a mature or differentiated cell. In some embodiments, the cells are incubated in a differentiation media for at least 1, 2, 3, 4, 5, 6, 7, 8, 9, 10, or more days. In some embodiments, the cells are incubated in a differentiation medium for between 1 and 4 days to allow for cell differentiation into a mature phenotype. In some embodiments, the cells are incubated in a differentiation medium for between 2 and 3 days to allow for cell differentiation into a mature phenotype. In some embodiments, the cultures have a TEER value of at least 100 Ω·cm² or greater after between 1 and 4 days in differentiation medium, or after between 2 and 3 days in differentiation medium. In some embodiments, the cultures have a TEER value of at least 200 Ω·cm² or greater after between 1 and 4 days in differentiation medium, or after between 2 and 3 days in differentiation medium. In some embodiments, the cultures have a TEER value of at least 250 Ω·cm² or greater after between 1 and 4 days in differentiation medium, or after between 2 and 3 days in differentiation medium. In some embodiments, the growth media is removed from the culture and differentiation media is added when the cells have a TEER value of at least 250 Ω·cm² or greater. In some embodiments, the cultures have a TEER value of at least 100, 200, 300, 400, 500, 600, 700, 800, 900, 1000, 1100, 1200, 1300, 1400, 1500 Ω·cm² or greater after between 1 and 4 days in differentiation medium, or after between 2 and 3 days in differentiation medium.

A mature or differentiated cell refers herein to a cell cultured according to the methods described herein that comprises one or more properties of an endogenous specialized or terminally differentiated cell. In some embodiments, a cell is identified as a mature or differentiated cell based on gene expression, protein expression, ion transport, amino acid transport, peptide transport, protein transport, monosaccharide transport, lipid transport, bile acid transport, organic molecule transport, small molecule transport, acid/base transport, polysaccharide metabolism, protein metabolism, peptide metabolism, lipid metabolism, small molecule metabolism, protein trafficking, protein localization, secretion of a hormone, secretion of metabolites, secretion of a peptide, secretion of monosaccharides, secretion of ions, secretion of small molecules, secretion of lipids, secretion of acid/base, apoptosis, rate of cell division, or TEER measurement.

In some embodiments, a 2D culture system is derived from a cell that was previously cultured in a 3D culture system (e.g., an organoid culture system). In some embodiments, an organoid culture system is derived from a primary human cell obtained from a subject's duodenum, jejunum, ileum, terminal ileum, and/or ascending, transverse, descending, and sigmoid colon. In some embodiments, the primary human cell is seeded in an organoid culture comprising a semi-solid matrix such as Matrigel. In some embodiments, the organoid culture further includes an organoid culture medium comprising EGF, a BMP inhibitor, an Lgr5 activator, a Wnt signaling agonist, nicotinamide, a TGFβ signaling antagonist, a ROCK inhibitor, and a p38/MAPK inhibitor. In some embodiments, the organoid culture media further comprises a GSK3 inhibitor. In some embodiments, the organoid culture media comprises EGF, Noggin, R-spondin 1, Wnt3a, A83-01, nicotinamide, Y-27632, SB202190, and CHIR99021. In some embodiments, the organoid culture media comprises EGF, Noggin, R-spondin 1, Wnt3a, A83-01, nicotinamide, Y-27632, and SB202190. In some embodiments, the organoid culture further comprises an organoid culture media comprising EGF, a BMP inhibitor, an Lgr5 activator, a Wnt signaling agonist, nicotinamide, a TGFβ signaling antagonist, and a p38/MAPK inhibitor. In some embodiments, the organoid culture media comprises EGF, Noggin, R-spondin 1, Wnt3a, A83-01, nicotinamide, and SB202190.

Any of the media described herein may further comprise a growth promoting factor and/or an antioxidant factor. In some embodiments, a growth promoting factor comprises additional compounds or molecules that promote the health and viability of the cultured cells. In some embodiments, the growth promoting factor is a supplement such as N2 or B27. In some embodiments, the growth promoting factor is a gastrin analog such as Leu-15 gastrin. In some embodiments, and the antioxidant factor may comprise N-acetylcysteine. In some embodiments, the media described herein may comprise a ROCK inhibitor. In some embodiments, a ROCK inhibitor may act as an apoptosis antagonist (e.g. one or more agents that inhibits, reduces, or prevents apoptosis). For example, in some embodiments, the ROCK inhibitors Y-27632 and thiazovivin can also act as apoptosis antagonists.

The amount or concentration of the agents added to the media described herein may be altered to optimize a particular characteristic or desired outcome of the culture. In some embodiments, the media described herein comprise about 1 ng/mL or more of EGF. In some embodiments, the media described herein comprise about 1 ng/mL to about 1000 ng/mL of EGF. In some embodiments, the media described herein comprise about 5 ng/mL to about 500 ng/mL of EGF. In some embodiments, the media described herein comprise about 1 ng/mL or more of a BMP inhibitor (e.g., Noggin). In some embodiments, the media described herein comprise about 1 ng/mL to about 1000 ng/mL of a BMP inhibitor. In some embodiments, the media described herein comprise about 10 ng/mL to about 500 ng/mL of a BMP inhibitor. In some embodiments, the BMP inhibitor is Noggin. In some embodiments, the media described herein comprise about 10 ng/mL to about 500 ng/mL of Noggin.

In some embodiments, the media described herein comprise about 1 ng/mL or more of an Lgr5 activator (e.g., R-spondin 1). In some embodiments, the media described herein comprise about 1 ng/mL to about 50 μg/mL of an Lgr5 activator. In some embodiments, the media described herein comprise about 50 ng/mL to about 20 μg/mL of an Lgr5 activator. In some embodiments, the Lgr5 activator is R-spondin 1. In some embodiments, the media described herein comprise about 50 ng/mL to about 20 μg/mL of R-spondin 1. In some embodiments, the media described herein comprise about 10 ng/mL of a Wnt signaling agonist (e.g., Wnt3a). In some embodiments, the media described herein comprise about 10 ng/mL to about 2000 ng/mL of a Wnt signaling agonist. In some embodiments, the media described herein comprise about 20 ng/mL to about 1000 ng/mL of a Wnt signaling agonist. In some embodiments, the Wnt signaling agonist is Wnt3a. In some embodiments, the media described herein comprise about 20 ng/mL to about 1000 ng/mL of Wnt3a.

In some embodiments, the media described herein comprise about 0.25 μM or more of a ROCK inhibitor (e.g., thiazovivin or Y-27632). In some embodiments, the media described herein comprise about 0.25 μM or more of a ROCK inhibitor. In some embodiments, the media described herein comprise about 0.25 μM to about 250 μM of a ROCK inhibitor. In some embodiments, the media described herein comprise about 1 μM to about 100 μM of a ROCK inhibitor. In some embodiments, the ROCK inhibitor is thiazovivin. In some embodiments, the media described herein comprise about 1 μM to about 100 μM of thiazovivin. In some embodiments, the media described herein comprise about 0.5 μM to about 25 μM of a ROCK inhibitor. In some embodiments, the ROCK inhibitor is Y-27632. In some embodiments, the media described herein comprise about 0.5 μM to about 25 μM of Y-27632.

In some embodiments, the media described herein comprise about 0.1 μM or more of a p38/MAPK inhibitor (e.g. SB202190, SB203580, or SB239063). In some embodiments, the media described herein comprise about 0.1 μM to about 100 μM of a p38/MAPK inhibitor. In some embodiments, the media described herein comprise about 1 μM to about 50 μM of a p38/MAPK inhibitor. In some embodiments, the media described herein comprise about 1 μM to about 50 μM of SB202190. In some embodiments, the media described herein comprise about M of a TGFβ signaling antagonist (e.g., A83-01, GW788388, LY364947, R268712, RepSox, SB431542, SB505124, or SB525334). In some embodiments, the media described herein comprise about 10 nM to 2000 nM of a TGFβ signaling antagonist. In some embodiments, the media described herein comprise about 100 nM to 1000 nM of a TGFβ signaling antagonist. In some embodiments, the TGFβ signaling antagonist is selected from A83-01, GW788388, LY364947, R268712, RepSox, SB431542, SB505124, and SB525334 In some embodiments, the media described herein comprise about 100 nM to 1000 nM of A83-01. In some embodiments, the media described herein comprise about 1 ng/mL of a differentiation promoting factor (e.g., BMP2, BMP4, BMP7). In some embodiments, the media described herein comprise about 1 ng/mL to about 2000 ng/mL of a differentiation promoting factor. In some embodiments, the media described herein comprise about 10 ng/mL to about 1000 ng/mL of a differentiation promoting factor. In some embodiments, the media described herein comprise about 10 ng/mL to about 1000 ng/mL of BMP2, BMP4, and/or BMP7.

In some embodiments, the methods described herein result in the generation of a 2D intestinal cell culture system, wherein the cell morphology, interaction, and function resemble those of a cell or population of cells in a primary intestinal tissue. In some embodiments, the cell culture methods described herein result in cultured cells with polarized membranes (e.g., apical and basolateral membranes), that form cell-cell contacts (e.g., gap junction, tight junctions, and/or desmosomes), and/or that transport ions, macromolecules, or other molecules between the apical and basolateral membranes. In some embodiments, the cultured cells demonstrate one or more of the following properties at a level, duration, and/or efficiency that is comparable to that of a cell or populations of cells in a primary intestinal tissue: expression of one or more proteins, expression of one or more polynucleotides, activity of a secondary messenger system, ion transport, amino acid transport, peptide transport, protein transport, monosaccharide transport, lipid transport, bile acid transport, organic molecule transport, small molecule transport, acid/base transport, polysaccharide metabolism, protein metabolism, peptide metabolism, lipid metabolism, small molecule metabolism, protein trafficking, protein localization, secretion of a hormone, secretion of metabolites, secretion of a peptide, secretion of monosaccharides, secretion of ions, secretion of small molecules, secretion of lipids, secretion of acid/base, post translational modification, changes in cell type, changes in number of cell types, apoptosis, rate of cell division, transepithelial electrical resistance, or composition of cell types.

In some embodiments, the methods provided herein result in the generation of a cultured population of cells suitable for testing the effects of one or more agents on intestinal epithelial cells (e.g., screening one or more agents). In some embodiments, these methods allow for the high-throughput screening of a plurality of agents. For example, in some embodiments, at least 100, 200, 300, 400, 500, 1000, 2000, 5000, 10,000 or more agents are screened. In such embodiments, the agents may comprise small molecules, proteins, nucleic acids, carbohydrates, and/or lipids. In some embodiments, the agent is an antibody, a natural or chemically modified polypeptide, a natural or chemically modified oligopeptide, a natural, unnatural, or chemically modified amino acid, a polynucleotide, a natural or chemically modified oligonucleotide, a natural or chemically modified mononucleotide, a lipopeptide, an antimicrobial, a small molecule, or a pharmaceutical molecule.

In some embodiments, cells cultured according to the methods described herein are contacted with one or more agents and a particular characteristic or property of a cell is measured. In some embodiments, the characteristic or property is expression of one or more proteins, expression of one or more genes, activity of a secondary messenger system, ion transport, amino acid transport, peptide transport, protein transport, monosaccharide transport, lipid transport, bile acid transport, organic molecule transport, small molecule transport, acid/base transport, polysaccharide metabolism, protein metabolism, peptide metabolism, lipid metabolism, small molecule metabolism, protein trafficking, protein localization, secretion of a hormone, secretion of metabolites, secretion of a peptide, secretion of monosaccharides, secretion of ions, secretion of small molecules, secretion of lipids, secretion of acid/base, post translational modification, changes in cell type, changes in number of cell types, apoptosis, rate of cell division, or transepithelial electrical resistance. Properties of a cell may be measured by means known in the art including but not limited to fluorescence microscopy, fluorometric assay, chromogenic assay, chemiluminescent assay, ion chromatography, HPLC, mass spectrometry, RNA sequencing, DNA sequencing, ELISA, enzyme assays, flow cytometry, FACS, TUNEL assay, viability assay, proliferation assay, chelation assay, immunocytochemistry, western blot analysis, qPCR analysis, radiometric changes, microarray analysis, or voltohm meter.

In some embodiments, the effect of an agent on a particular property or characteristic is assessed by comparing the property of a cell contacted with the agent to the property of a control cell or control cell population (e.g., a reference standard). In some embodiments, a reference standard is determined from a cell that has not been contacted with the agent. In some embodiments, a reference standard is determined by measuring a particular property of a cell prior to contacting the cell with the agent (e.g., a baseline measurement). In some embodiments, a reference standard is determined by measuring the property of a cell that has been contacted with a control agent (e.g., a positive or negative control). In some embodiments, the control agent is a negative control agent such as a solution, solvent, or media (e.g., a vehicle control). In some embodiments, a control agent is a positive control agent, wherein the positive control agent exerts a known effect on a particular property of a cell (e.g., a positive control agent increases or decreases a property of a cell).

In some embodiments, the methods provided herein result in the generation of a cultured population of cells suitable characterizing the site of action of an agent. As used herein, a “site of action” refers to a cellular site wherein an agent exerts a particular effect. In some embodiments, a site of action may refer to a particular fraction of a cell, such as the plasma membrane (e.g., an intracellular or extracellular region of an apical or a basolateral membrane), the cytosol, and/or the nucleus. In some embodiments, a site of action may refer to a cellular organelle, such as a ribosome, a proteasome, a nucleosome, a centriole, a Golgi apparatus, a mitochondria, and/or an endoplasmic reticulum. In some embodiments, a site of action may refer to a site located between two or more cells (e.g., the intercellular space of a gap junction). In some embodiments, a site of action may refer to a particular cellular molecule such as a cell surface protein, a secreted protein (e.g., a cytokine, a chemokine, or an enzyme), a nuclear protein (e.g., a transcription factor), or a nucleic acid (e.g., DNA or RNA).

In some embodiments, the site of action of a particular agent is known. In some embodiments, the site of action of a particular agent is not known. In such embodiments, the present disclosure provides methods for identifying the site of action of a particular agent. In some embodiments, the site of action of a particular agent is identified by contacting the cultured cells with an agent in the presence of a competitor agent, wherein the site of action of the competitor agent is known. If the agent does not modify a particular property in the presence of the competitor agent, but does modify the property in the absence of the competitor agent, this indicates that the agent and competitor agent modify the same, or overlapping, sites of action.

In some embodiments, an agent comprises a detectable label such as a fluorophore, a radioisotope, and/or an oligonucleotide. In some embodiments, the site of action of a particular agent is determined by detecting the proximity of the agent to a protein or other component of a particular site of action. In such embodiments, the agent and the protein or component each comprise a distinct detectable label. In such embodiments, detection of the agent and protein or compound may occur by PLA, FRET, or other means known in the art. In some embodiments, the agent is a DRA inhibitor, a TGR5 inhibitor, or a TGR5 activator.

In a certain embodiment, there is provided a method for inhibiting the transport of sodium across a two-dimensional (2D) monolayer cell culture as described herein, comprising contacting the apical side of said monolayer with the compound

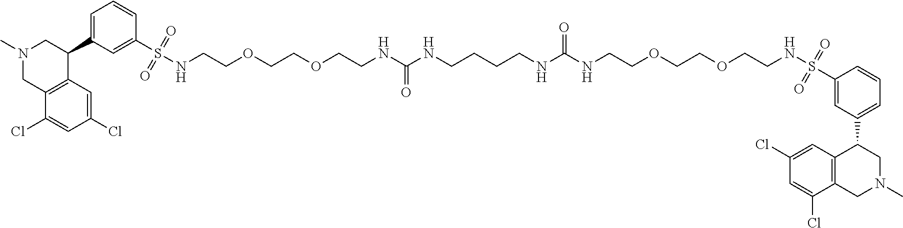

or a pharmaceutically acceptable salt thereof.

In a certain embodiment, there is provided a method for inhibiting the transport of phosphate across a two-dimensional (2D) monolayer cell culture as described herein, comprising contacting the apical side of said monolayer with the compound

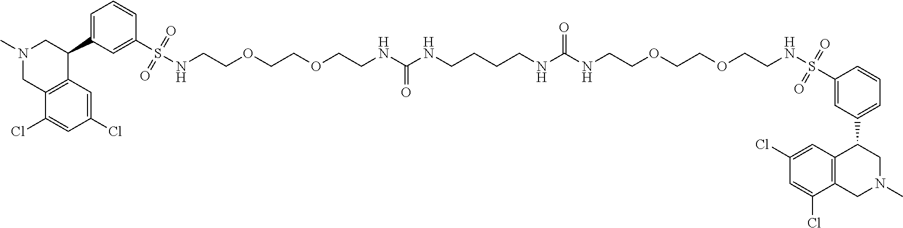

or a pharmaceutically acceptable salt thereof.

### EXAMPLES

The following examples for the purpose of illustrating various embodiments of the disclosure. The present examples, along with the methods described herein, are exemplary, and are not intended as limitations on the scope of the disclosure. Alterations, modifications, and other changes to the described embodiments which are encompassed within the spirit of the disclosure as defined by the scope of the claims are specifically contemplated.

### Example 1—Culture of Primary Intestinal Cells

### Reagents

Growth media and growth factors were based on published protocols (Sato et al. (2009) Nature 459(7244):262-265; Sato et al. (2011) Gastroenterology 141(5):1762-1772; Wang et al. (2013) Lab Chip 13(23):4625-4634). Basal media (BM) consisted of advanced DMEM/F12 containing 10 mM HEPES (Invitrogen, 15630-080), 1:100 Glutamax (Invitrogen, 35050-061), and 1:100 penicillin/streptomycin (Invitrogen, 15140-122). Supplemented basal media (SBM) for murine cultures contained 1:100 N2 (Invitrogen, 17502-048), 1:50 B27 (Invitrogen, 12587-010), 1 mM N-acetylcysteine (Sigma, A9165). SBM for human cultures additionally contained 10 nM [Leu15]-gastrin I (Sigma, G9145). Growth factors used were 50 ng/mL mouse EGF (Peprotech, 315-09), 100 ng/mL mouse noggin (Peprotech, 250-38), 500 ng/mL human R-spondin 1 (R&D, 4645-RS), 100 ng/mL mouse Wnt-3a (R&D, 1324-WN), 20 μM Y-27632 (Tocris, 1254), 2.5 μM thiazovivin (Tocris, 3845), 10 mM nicotinamide (Sigma, N0636), 500 nM A83-01 (Tocris, 2939), 10 μM SB202190 (Tocris, 1264), human BMP-2 (Peprotech, 120-02C), mouse BMP-4 (Peprotech, 315-27), and human BMP-7 (Peprotech, 315-27).

Transwells were 0.4 μm pore polyester membrane 24-well Transwell inserts (Corning) or 1 μm pore polyester membrane 96-well Transwell inserts (Corning).

The following Wnt pathway inhibitors were evaluated during culture development and optimization: Cardionogen (Tocris), iCRT14 (Tocris), IWP4 (Tocris), KY02111 (Tocris), FH535 (Tocris), and JW67 (Tocris).

Antibodies for lineage staining were rabbit anti-lysozyme (Dako, A0099), rabbit anti-chromogranin A (Abcam, ab15160), rabbit anti-mucin 2 (Santa Cruz, SC-15334), rabbit anti-NHE3 (Novus, NBP1-82574), and rat anti-CD13 (MCA2183GA).

### Murine Intestinal Crypt Isolation

The process of isolating mouse intestinal crypts from the colon and small intestine was based on a modification of published protocols (Gracz et al., (2012) Methods Mol Biol 879:89-107). Briefly, intestines were isolated from 6-12-week-old C57BL/6 mice, rinsed with DPBS using a gavage needle, and cut into different segments. For mouse colonic cultures, the colon was divided in to distal and proximal sections for respective cultures. For mouse proximal colonic cultures, the proximal one-third of the colon, excluding the cecum, was used. For small intestine cultures, the small intestine was divided into duodenum, jejunum, and ileum. For duodenum cultures, the proximal 4-6 cm of the small intestine was used. For jejunum and ileum cultures, the remaining small intestine was cut in half. The proximal half was used for jejunum cultures and the distal half was used for ileum cultures. An additional 3-4 cm of tissue was trimmed from the proximal and distal ends of the jejunum and from the proximal end of the ileum.

The segments were then cut longitudinally and stored in DPBS on ice before crypt isolation. For small intestine cultures, the duodenal, jejunal, and ileal segments were gently scrapped to remove villi. Each tissue segment was placed in ice-cold crypt isolation buffer 1 (CIB1) containing DPBS with 30 mM EDTA and 1.5 mM DTT, and incubated on ice for 20 minutes. The tissue was then transferred to pre-heated (to 37° C.) crypt isolation buffer 2 (CIB2) containing DPBS with 30 mM EDTA and incubated for 10 minutes in a water bath at 37° C. The tube was then removed and shaken vigorously for 30 seconds to release the crypts. The tissue was transferred to a second tube containing pre-heated CIB2 and shaken vigorously for 30 seconds or until no more crypts appeared to be released from the tissue.

Crypts were washed with basal media (BM) and the two aliquots for each tissue segment were combined in one tube. Crypts were centrifuged and re-suspended in 3 mL of dispase solution (Invitrogen, 17105-041) pre-heated to 37° C. The solution was continuously pipetted for approximately 8 minutes for distal colon and 5 minutes for proximal colon and all small intestine sections. The dispase solution was diluted with 5 mL basal media (BM) and then centrifuged for 3 minutes at 1,000×g. The pellet was resuspended in 5 mL BM and the cell suspension was filtered through a 70 μm cell strainer. The strainer was washed once with 5 mL BM to increase the yield of cells.

### Cell Lineage Staining

The cell lineage antibody staining protocol was performed at ambient temperature. Monolayer cultures were fixed in 100 μL of 2% paraformaldehyde for 5 minutes. The wells were washed twice with DPBS and once with blocking buffer (PBS containing 1% BSA and 0.5% saponin). The wells were blocked and permeabilized in blocking buffer for 30 minutes. Primary antibodies (anti-Muc2 1:1,000; anti-Chga 1:100; anti-Lysozyme 1:250; anti-NHE3 1:200; anti-CD13 1:250) were diluted in blocking buffer and incubated for 2 hours. The wells were then washed three times with blocking buffer. Secondary antibody (donkey anti-rabbit Alexa Fluor 555 Invitrogen), diluted 1:2,000 in blocking buffer, was incubated for 30 minutes. The wells were washed twice with blocking buffer and once with DPBS and then incubated with 2.5 μg/mL DAPI for 5 minutes. The wells were washed twice with DPBS. The Transwells were excised and mounted on microscope slides using ProLong Gold antifade reagent (Thermo Fisher).

RNA Isolation from Cell Cultures, Sequencing and Analysis

RNA was isolated from monolayer cultures using RNeasy (Qiagen) kits. Briefly, monolayers were directly lysed by adding RLT Plus containing 2-mercaptoethanol to the apical side of the Transwell and scraping the bottom of the well with the 1 mL pipette tip. RNA isolation was performed according to the manufacturer's instructions using RNeasy spin columns.

Approximately 1 μg of the entire isolated RNA was used to prepare each RNA sequencing library. RNA libraries were prepared using the TruSeq Stranded mRNA library preparation kit (Illumina). Most sequencing data were obtained using a NextSeq500 sequencer (Illumina) (2×75 bp, ˜ 30-40 million reads per sample) and some sequencing was performed using a HiSeq sequencer (Illumina) (2×125 bp, ˜ 25-30 million reads per sample). Transcript counts were obtained using the RNA Express application (RNA-Seq reads aligned with the STAR aligner) on the Illumina BaseSpace website.

Gene lists used to make heat maps were obtained by determining the most differentially expressed genes in a given intestinal tissue segment and applying that gene list to monolayer culture transcript data. For mouse colon, genes were selected as having greater than five-fold increase in expression in proximal vs. distal colonic tissue, and vice versa, with counts above 100. For multiple tissue comparison, genes were selected based on having greater than five-fold increase in expression when comparing the tissue segment with the sum of the other tissue segments with counts above 100. Gene counts were normalized to the highest expressed value for tissues and cultures. Heat maps and clustering were generated using the heatmap.2 function in R.

Pearson correlation coefficients were determined in Microsoft Excel using the Pearson function comparing the transcript count correlation for all genes between two data sets.

RNA Isolation from Tissue, Sequencing and Analysis

RNA for sequencing was isolated from mouse small intestinal villi and crypts, and colon crypts. Mouse small intestines were isolated and cut into different segments as described for monolayer growth above. The segments were then cut longitudinally and gently scraped with a microscope slide to remove villi, which were placed in RLT Plus with 2-mercaptoethanol and vortexed to lyse the cells. Crypts were isolated from the remaining intestinal tissue and pelleted as described for the small intestine above, before being placed in RLT Plus with 2-mercaptoethanol and vortexed to lyse the cells.

Mouse colons were isolated and cut into different segments as described for monolayer growth above. The segments were then cut longitudinally. Crypts were isolated and pelleted as described for the colon above, before being placed in RLT Plus with 2-mercaptoethanol and vortexed to lyse the cells.

Human intestinal epithelial cells were obtained from biopsies and pelleted as described for human organoid growth above. Cells were placed in RLT Plus with 2-mercaptoethanol and vortexed to lyse the cells.

RNA was isolated from lysed cells using RNeasy spin columns and RNA sequencing was performed as described above.

### Ion-Transport Analysis

Ion transport was determined by sampling the apical and basolateral chambers of the Transwell and analyzing these samples on an ion chromatography system (Thermo Fisher ICS-3000 or ICS-5000+) coupled with conductivity detectors. Chromatographic separation of cations was performed using an IonPac CS12A (Dionex) 2×250 mm analytical column with an isocratic elution using 25 mM methanesulfonic acid. Chromatographic separation of anions was performed using an IonPac AS18 (Dionex) 2×250 mm analytical column with an isocratic elution using 35 mM potassium hydroxide. Concentrations were calculated relative to a standard curve for each analyte ion based on retention time and peak area.

### Mouse Distal Colon K+ Transport Screening Assay

An assay was established to measure K⁺ transport in mouse distal colonic cultures. Mouse distal colonic monolayer cultures were prepared in 96-well Transwells according to protocol and were used for assay on day 6, 7, or 8 depending on the TEER value of the culture and its water-absorption phenotype. Wells with a TEER value below 450 Ω·cm² were not used for assay. For screening, apical media consisted of 500 μM rubidium chloride and 30 μM compound in DMEM/F12 with HEPES and glutamine (Invitrogen). Basolateral media consisted only of DMEM/F12 with HEPES and glutamine. The apical and basolateral sides of plates were washed three times with DMEM/F12 with HEPES and glutamine.

To start the assay, 80 μL of apical media was added to triplicate wells per compound and 130 μL of basolateral media was added to the basolateral compartment. Transwells were placed in an incubator at 37° C., 5% CO₂ for 3 hours, and then 15 μL of apical media was removed from each well and diluted in 485 μL of deionized water. K⁺ and rubidium ion concentrations were determined using ion chromatography. TEER values were measured at the end of each run to determine which compounds caused a drop in TEER. Rubidium concentration was also used as a measure of tight junction integrity. Each plate contained at least six wells containing 200 μM vanadate as a positive control for inhibition of K⁺ transport and 15 wells of 0.3% DMSO vehicle as a negative control. On each plate, K⁺ transport was normalized to the vanadate control. Compounds that inhibited K⁺ transport greater than 3 SDs from the mean were considered positive except for compounds that resulted in a decrease in TEER and increase in rubidium transport above control values owing to increased paracellular leakage. Hits were repeated, and those that remained positive were dose-titrated.

Compounds from the Tocriscreen Compound Library Collection (Tocris) and LOPAC 1280 (Sigma) were used in the screen, together with several additional inhibitors of the Na⁺/K⁺-ATPase that were not in either library (bufalin, cinobufagin, resibufogenin, proscillaridin A, digitoxigenin, and digitoxin).

### Example 2—Characterization of Murine Colon Monolayer Cultures

Two dimensional cell cultures from mouse intestines were generated beginning with freshly isolated crypts, isolated as described in Example 1. Culture conditions were based on the growth factor combination used in the growth media used for three-dimensional mouse colonic organoids: Wnt3a (W), epidermal growth factor (E), noggin (N), and R-spondin 1 (R) (murine colonic growth media, WENR).

Briefly, after cell isolation from intestinal crypt, cells were transferred to a new 15 mL conical centrifuge tube and centrifuged for 3 minutes at 1,000×g. The pellet was resuspended in seeding media comprising Wnt3a (W), epidermal growth factor (E), noggin (N), R-spondin 1 (R), and Y27632 (Y) (murine colonic seeding media, WENRY) at 0.5×10⁶ cells per mL. For culture in 24-well Transwell plates, 200 μL of cell suspension was added to the apical compartment of the transwells (100,000 cells per well) and 600 μL of WENRY media was added to the basolateral side of transwells. For culture in 96-well Transwells, 80 μL of cell suspension was added to the apical compartment of the transwells (40,000 cells per well) and 200 μL of WENRY media was added to the basolateral side the transwells. Cultures were incubated at 37° C., 5% CO₂.

On day 1 after seeding, 100 μL and 40 μL of BM was gently pipetted onto the apical side of 24-well and 96-well Transwells, respectively, in order to resuspend non-adherent cells. On day 2, WENR media was added to the cultures. On day 4, confluence of the cultures was assessed by measuring the TEER using Millicell ERS2 (Millipore). Confluent cells will have TEER values greater than 500Ω, optionally between 5000 and 13000Ω. If the cultures were confluent based on TEER readings, differentiation media comprising epidermal growth factor (E) and noggin (N) (murine colonic differentiation media, EN) was added to the basolateral side of the transwell and SBM was added to the apical side of the transwell. If cultures were not confluent, fresh WENR media was added on day 4, followed by addition of EN media to the basolateral side of the transwell and SBM to the apical side of the transwell on Day 5. Cells were typically differentiated by day 7 or day 8 with TEER values between 6000 and 15000Ω. Descriptions of the media and buffers used for colonic cell isolation and culture are provided in Table 1.

TABLE 1
Medias and Buffers for Murine 2D Colonic Cultures

| Solution Type | Component |
|---|---|
| Basal Media (BM) | Advanced DMEM/F12 |
| | 10 mM HEPES |
| | 1:100 Glutamax |
| | 1:100 Penicillin/Streptomycin |
| Supplemented Basal | 50 mL BM |
| Media (SBM) | 1 mL B27 Supplement |
| | 500 μL N2 Supplement |
| | 100 μL of 500 mM N-acetylcysteine |
| Colonic Growth | SBM |
| Media (WENR) | 1:1000 of 50 μg/mL murine EGF |
| | 1:1000 of 100 μg/mL murine Noggin |
| | 1:1000 of 500 μg/mL human RSpondin-1 |
| | 1:500 of 50 μg/mL murine Wnt3a |
| Colonic Seeding | SBM |
| Media (WENRY) | 1:1000 of 50 μg/mL murine EGF |
| | 1:1000 of 100 μg/mL murine Noggin |
| | 1:1000 of 500 μg/mL human RSpondin-1 |
| | 1:500 of 50 μg/mL murine Wnt3a |
| | 1:1000 of 20 mM Y27632 |
| Colonic Differentiation | SBM |
| Media (EN) | 1:1000 of 50 μg/mL murine EGF |
| | 1:1000 of 100 μg/mL murine Noggin |
| Crypt Isolation | DPBS |
| Buffer 1 (CIB1) | 30 mM EDTA |
| | 1.5 mM DTT |
| Crypt Isolation | DPBS |
| Buffer 2 (CIB2) | 30 mM EDTA |
| Dispase Solution | 8 mg dispase |
| | 10 mL HBSS |
| | 10 mM HEPES |

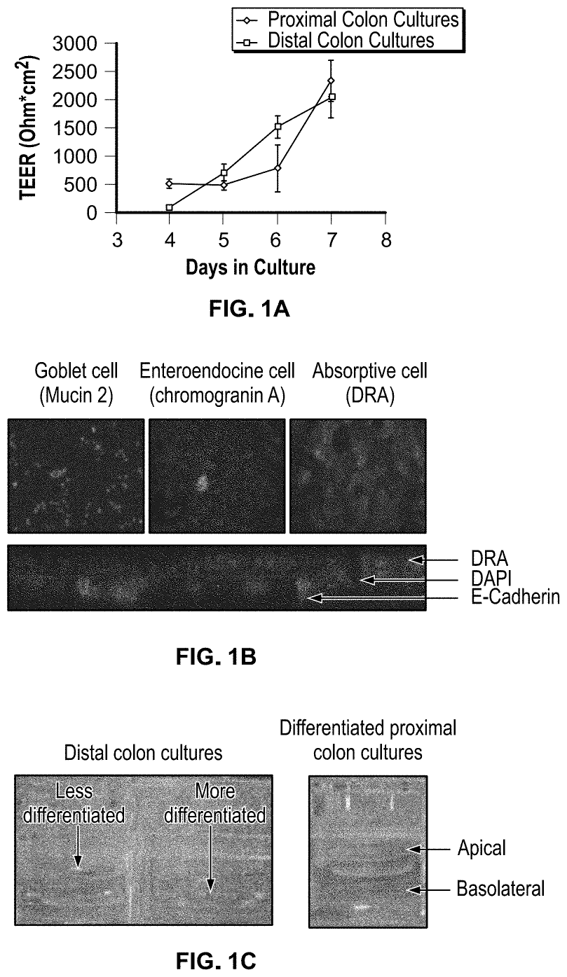

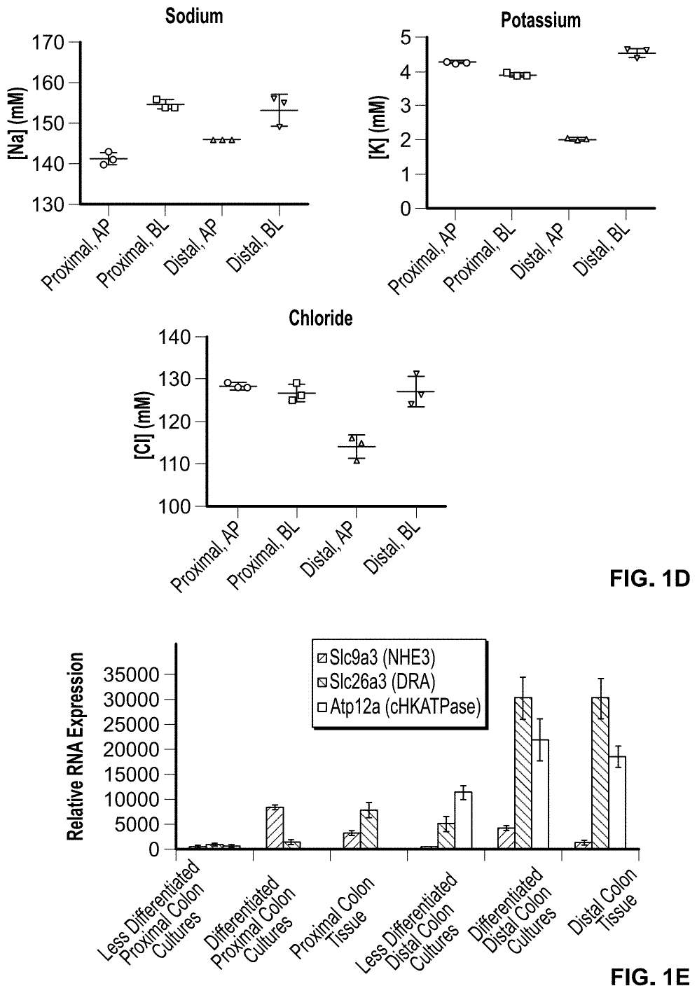

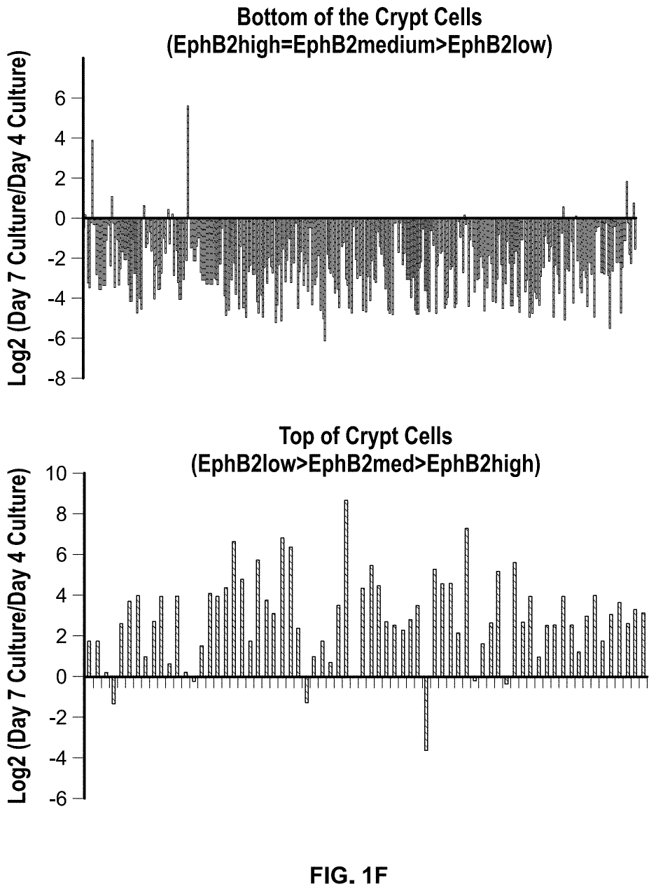

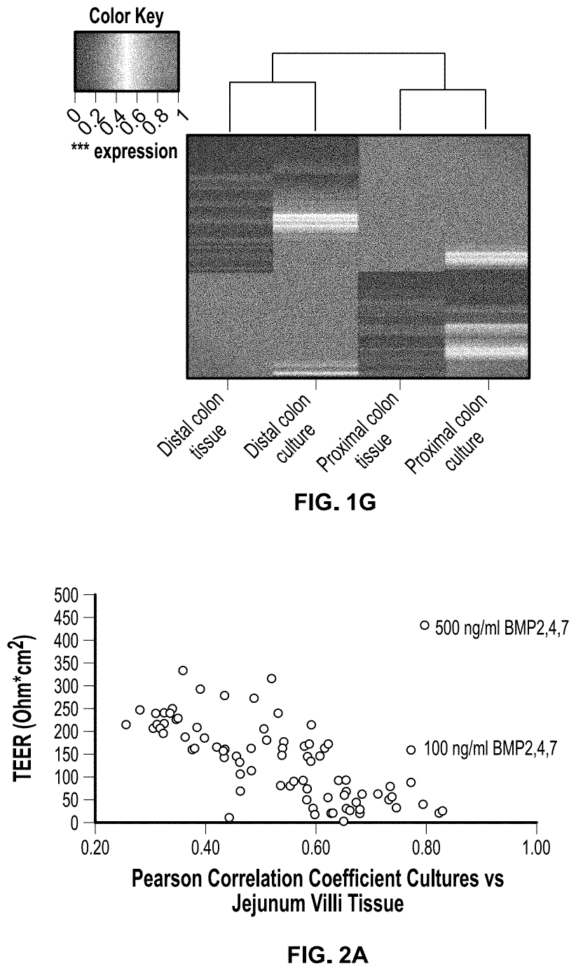

Using WENR growth media, mouse colonic cultures achieved confluence and yielded improving transepithelial electrical resistance (TEER) values over time, suggesting the formation of tight junctions (FIG. 1A). Although cultures spontaneously differentiated upon forming a confluent monolayer, presumably owing to contact inhibition, removal of Wnt3a and R-spondin1 accelerated differentiation, and TEER values exceeded 2,000 Ω·cm² under these conditions.

Cultures were stained for markers of the major cell lineages in the mouse colon in order to confirm differentiation into absorptive, goblet, and enteroendocrine cell lineages (FIG. 1). Confocal microscopy imaging confirmed cell polarization based on the apical localization of the chloride (Cl⁻)/bicarbonate ion transporter DRA (Slc26a3) relative to the cell nucleus and to basolateral cell junctions (FIG. 1).

After differentiation, mouse distal colonic cultures showed a phenotype characterized by rapid absorption of water from the apical compartment into the basolateral compartment (FIG. 1C). In contrast, differentiated mouse proximal colonic cultures showed reduced water absorption compared with distal colonic cultures but displayed an apically directed acid-secretion phenotype as demonstrated by a yellow color of pH-sensitive phenol red dye in the apical media (indicating acidic pH) and a pink color in the basolateral media (indicating neutral pH) (FIG. 1C). Ion chromatography analysis of the media in both compartments showed net sodium ion (Na⁺), K⁺, and Cl⁻ absorption from the apical compartment into the basolateral compartment in the distal colonic cultures and net Na⁺ absorption in the proximal colonic cultures (FIG. 1D). Taken together, these phenotype and ion-transport measurements demonstrate functional ion transport as well as ion-transporter expression patterns in murine colonic cultures.

Further analyses were performed to confirm the expression of ion transporters in the proximal and distal sections of the colon. Global transcript expression of three ion transporters, NHE3 (Slc9a3), DRA(Slc26a3), and colonic H⁺/K⁺-ATPase (ATP12A) was obtained using RNA sequencing from freshly isolated colonic crypts, less-differentiated mouse colonic cultures (taken on day 4), and more-differentiated colonic cultures (taken on day 7). The apical Na*/hydrogen ion (H⁺) exchanger, NHE3 (Slc9a3), is expressed endogenously at high levels in the proximal and mid colon and is responsible for absorption of Na⁺ and excretion of H⁺ (Talbot et al., (2010), Am J Physiol Gastrointest Liver Physiol 299(2):G358-367). DRA is endogenously expressed in the mid and distal colon and is responsible for the absorption of Cl⁻ in exchange for bicarbonate ions (Talbot et al., (2010), Am J Physiol Gastrointest Liver Physiol 299(2):G358-367). Finally, the apical colonic H⁺/K⁺-ATPase ATP12A is endogenously expressed exclusively in the distal colon and functions to absorb K⁺ and secrete H⁺ (Sangan et al., (1997), Am J Physiol 272(2 Pt 1):C685-696). Expression patterns of NHE3, DRA, and colonic H⁺/K⁺-ATPase in the murine colonic cultures were consistent with the observed phenotype, with NHE3 expressed in both proximal and distal colonic cultures and DRA and colonic H⁺/K⁺-ATPase showing higher expression levels in the distal than in the proximal colonic cultures (FIG. 1E).

Differential gene expression patterns in day 4 versus day 7 mouse colonic cultures were compared with previously reported lists representing the most differentially expressed genes from less differentiated (bottom-of-the-crypt) and more differentiated (top-of-the-crypt) colonic cells isolated from tissue, sorted based on Ephb2 expression (Merlos-Suarez et al., (2011) Cell Stem Cell 8(5):511-524). The results suggest that the gene expression pattern in day 4 mouse colonic cultures is consistent with less differentiated, or bottom-of-the-crypt, colonic cells, while the gene expression pattern in day 7 colonic cultures is consistent with more differentiated, or top-of-the-crypt, colonic cells (FIG. 1F).

RNA sequencing was used to evaluate whether the cultures maintained segment-specific gene expression patterns, and the expression of genes with at least five-fold differential expression between mouse proximal and distal colonic tissues was compared with the relative expression of these genes in the cultures. The cultures clustered unsupervised with the intestinal tissue segment of origin, and relative gene expression patterns in the cultures were similar to those in the corresponding tissue segments (FIG. 1G).

### Example 3—Characterization of Murine Small Intestine Monolayer Cultures

Unlike mouse colonic cultures, mouse jejunal cultures were unable to grow to confluence in either the mouse colonic culture condition using WENR or the standard three-dimensional organoid media using ENR (EGF (E), noggin (N), R-spondin 1(R)). Experiments were performed to optimize seeding and growth culture conditions for mouse jejunal cultures. Two modifications of the mouse colonic culture conditions were necessary for the cells to grow to confluence reproducibly. First, the rho-associated protein kinase (ROCK) inhibitor Y-27632 (Y), which is typically added only for the first 2 days after seeding the cells in order to minimize anoikis, was required throughout the duration of the culture. Secondly, Wnt3a concentration needed to be increased from the standard concentration of 100 ng per mL; 250 ng per mL was chosen as a concentration that reproducibly produced confluent cultures with acceptable TEER values (i.e. greater than 450 Ω·cm²).

Briefly, two dimensional cell cultures from mouse intestines were generated beginning with freshly isolated crypts, isolated as described in Example 1. After cell isolation from intestinal crypt, cells were transferred to a new 15 mL conical centrifuge tube and centrifuged for 3 minutes at 1,000×g. The pellet was resuspended in seeding media comprising Wnt3a (W), epidermal growth factor (E), noggin (N), R-spondin 1 (R), and Y27632 (Y) (murine small intestine seeding and growth media, W_{2.5}ENRY) at 0.5×10⁶ cells per mL. For culture in 24-well Transwell plates, 200 μL of cell suspension was added to the apical compartment of the transwells (100,000 cells per well) and 600 μL of W_{2.5}ENRY media was added to the basolateral side of transwells. For culture in 96-well Transwells, 80 μL of cell suspension was added to the apical compartment of the transwells (40,000 cells per well) and 200 μL of W_{2.5}ENRY media was added to the basolateral side the transwells. Cultures were incubated at 37° C., 5% C02.

On day 2, fresh W_{2.5}ENRY was added to the cultures, and was replaced on day 4. Confluence of the culture was assessed by measuring the TEER. Confluent cells had TEER values between 700 and 1200Ω in 24 well transwells. Cultures were differentiated on day 5 using SBM supplemented with EGF, thiazovivin, and 300 ng/mL BMP4.

Descriptions of the media and buffers used for small intestine cell isolation and culture are provided in Table 2.

TABLE 2
Medias and Buffers for Murine 2D Small Intestine Cultures

| Solution Type | Component |
|---|---|
| Basal Media (BM) | Advanced DMEM/F12 |
| | 10 mM HEPES |
| | 1:100 Glutamax |
| | 1:100 Penicillin/Streptomycin |
| Supplemented Basal | 50 mL BM |
| Media (SBM) | 0.5 mL N2 Supplement |
| | 1 mL B27 Supplement |
| | 100 μL of 500 mM N-acetylcysteine |
| Small Intestine Seeding | SBM |
| Media and Growth Media | 1:1000 of 50 μg/mL murine EGF |
| (W_{2.5}ENRY) | 1:1000 of 100 μg/mL murine Noggin |
| | 1:1000 of 500 μg/mL human RSpondin-1 |
| | 1:200 of 50 μg/mL murine Wnt3a |
| | 1:1000 of 20 mM Y27632 |
| Small Intestine | SBM |
| Differentiation Media | 1:1000 of 50 μg/mL murine EGF |
| (ET + BMP) | 1:2000 of 5 mM thiazovivin |
| | 1:1000 of 300 μg/mL murine BMP4 |
| Crypt Isolation Buffer 1 | DPBS |
| | 30 mM EDTA |
| | 1.5 mM DTT |
| Crypt Isolation Buffer 2 | DPBS |
| | 30 mM EDTA |
| Dispase Solution | 8 mg dispase |
| | 10 mL HBSS |
| | 10 mM HEPES |

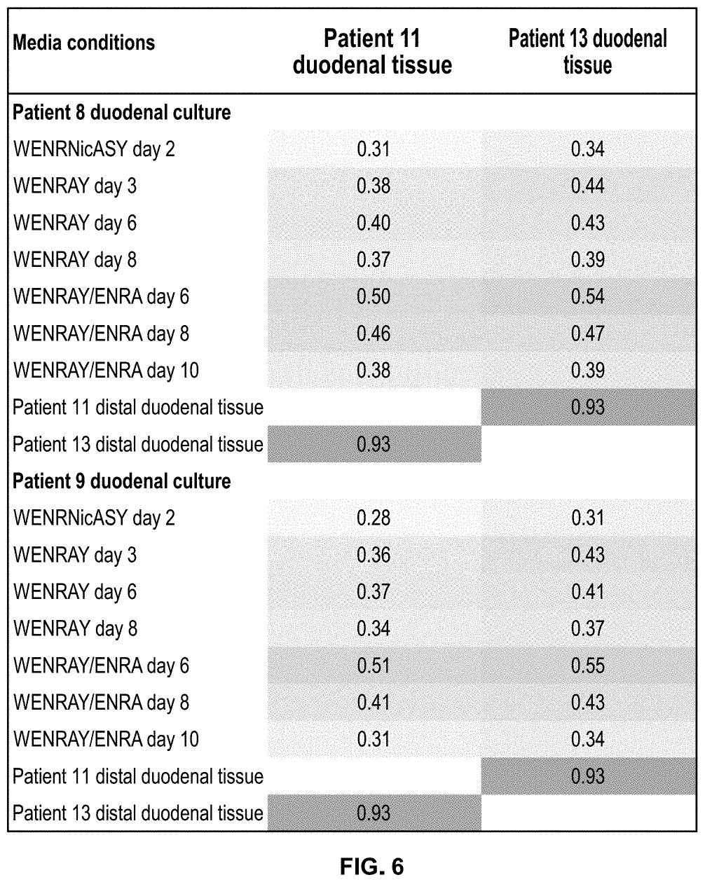

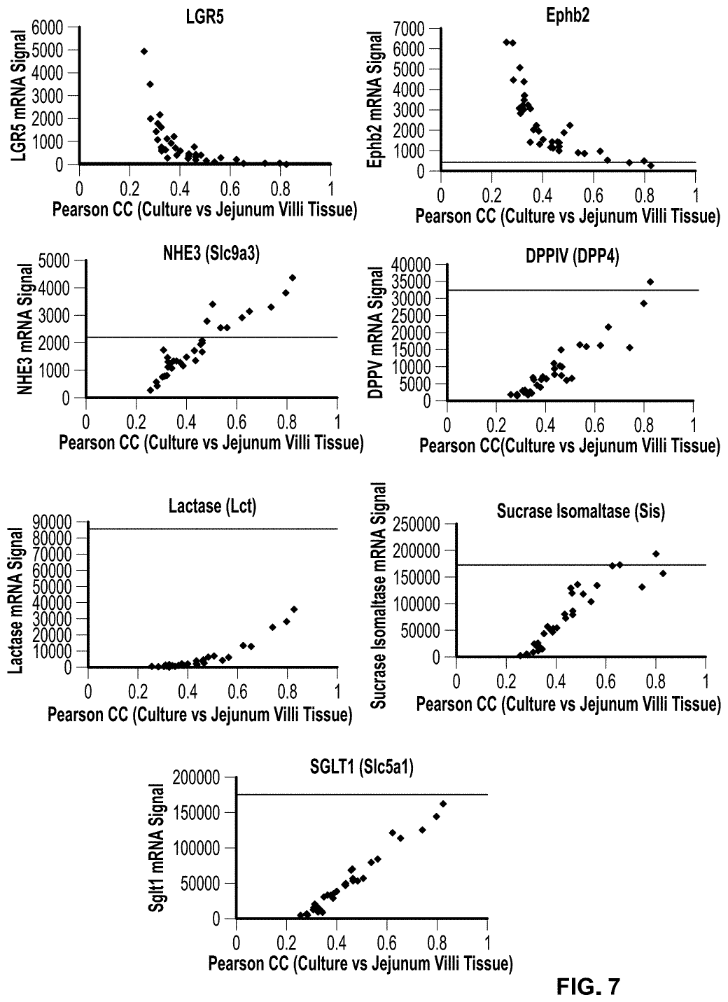

Despite achieving confluence, murine jejunal cultures did not display a strong functional phenotype, and it was unclear whether these cultures represented well-differentiated villus-like cells or less-differentiated crypt-like cells. Therefore, global gene expression by RNA sequencing was obtained for 36 culture conditions, taken 4, 6, and 8 days after seeding the cultures, and compared with the gene expression profiles of mouse jejunal villi and crypts. To simplify analysis and to account for changes in global gene expression rather than relying on a handful of marker genes, the Pearson correlation coefficient (PCC) was calculated for the relationship between the gene expression profiles of cultured cells and of mouse jejunal villus cells. Use of the PCC as a quantitative measurement of similarity was verified by comparing the correlation coefficients for the expression profiles of genes that are markers of either undifferentiated cells (Lgr5, Ephb2) or differentiated cells (lactase (Lct), sucrose-isomaltase (Sis), Sglt1(Slc5a1), DPP IV (Dpp4), and NHE3 (Slc9a3)). The resulting strong negative correlation with undifferentiated cell markers and positive correlation with differentiated cell markers validated this approach of optimizing growth conditions for differentiated small intestinal cultures (FIG. 6).

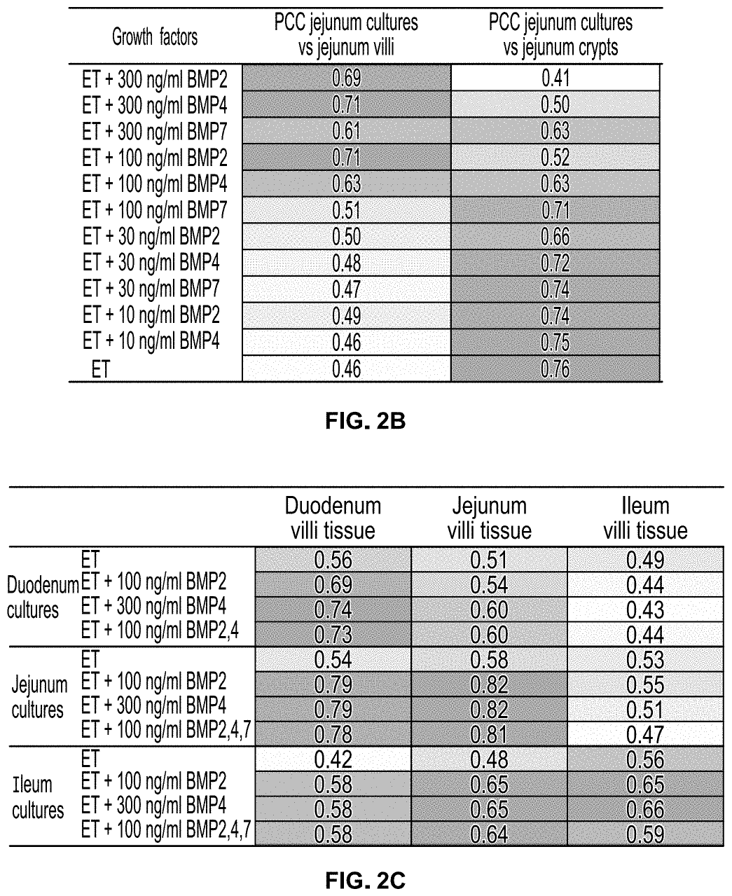

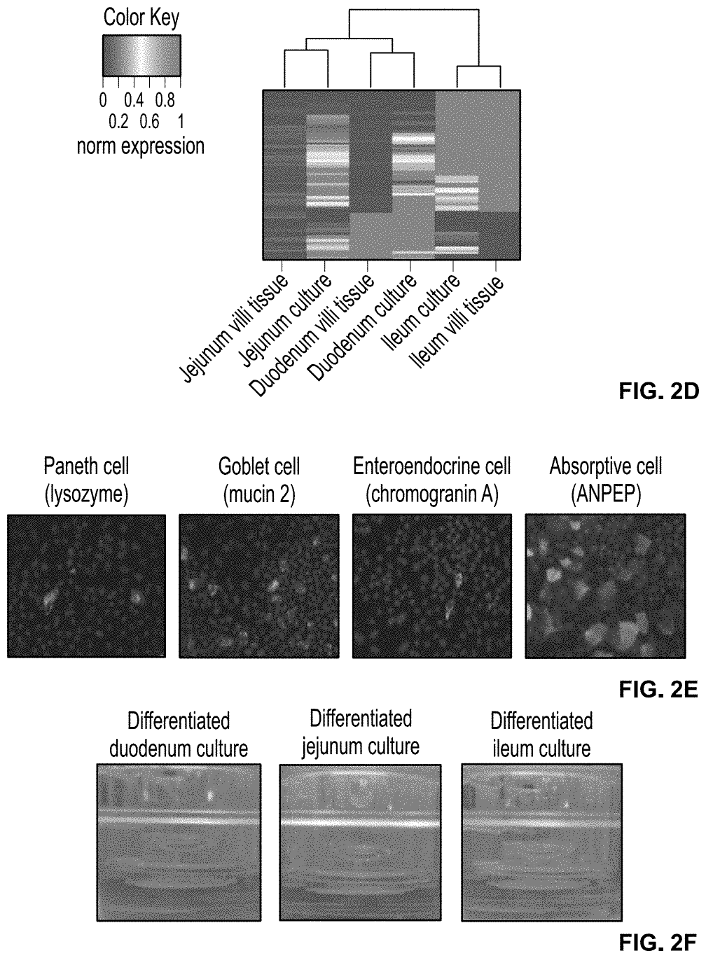

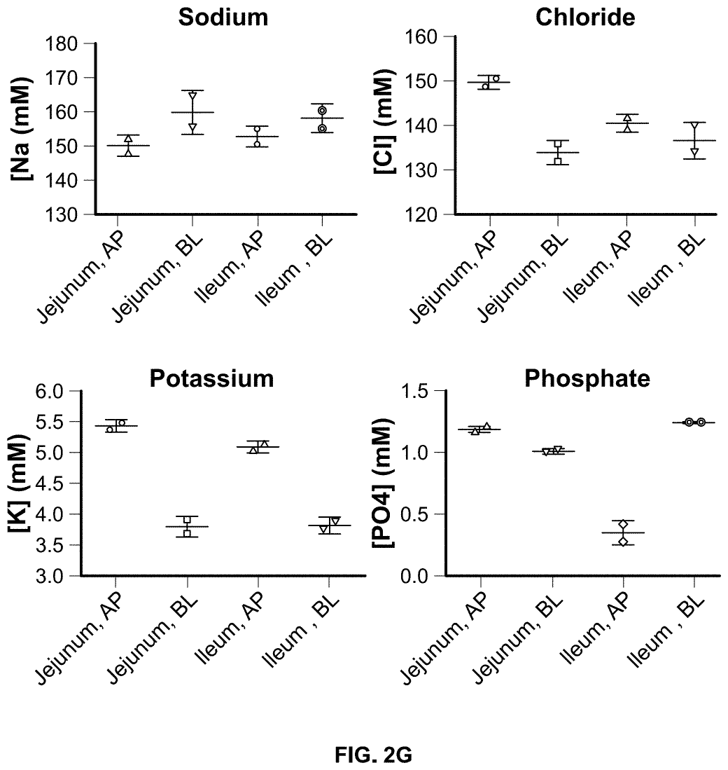

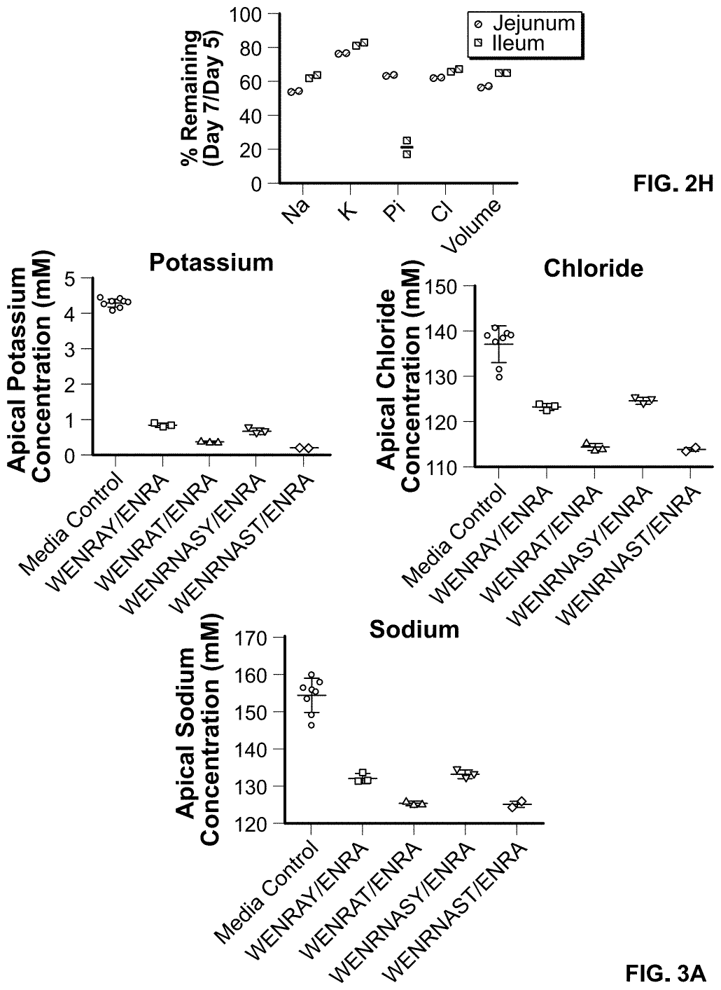

The expression profiles of several samples from day 8, showed a strong correlation with mouse jejunal villi gene expression (PCC up to approximately 0.8). However, these cultures contained visual gaps in the monolayer and TEER values were near background levels, despite having reaching confluence and demonstrating healthy TEER values earlier in the culture. Therefore, a second set of gene expression data using 48 culture conditions was obtained in which measures were taken to rapidly differentiate the cultured cells after they had reached confluence. A panel of small-molecule Wnt pathway inhibitors and a mixture of bone morphogenetic proteins (BMPs) 2, 4, and 7 were tested as a means to induce rapid differentiation. Plotting the PCC against TEER value for these samples and for the 36 samples from the previous experiment confirmed the trend of reduced TEER values in cultures that had the highest correlation with jejunal villi, except for cultures treated with either 100 ng/mL or 500 ng/mL BMPs 2, 4, and 7, which both showed a strong correlation with jejunal villi and maintained healthy TEER values (FIG. 2A).

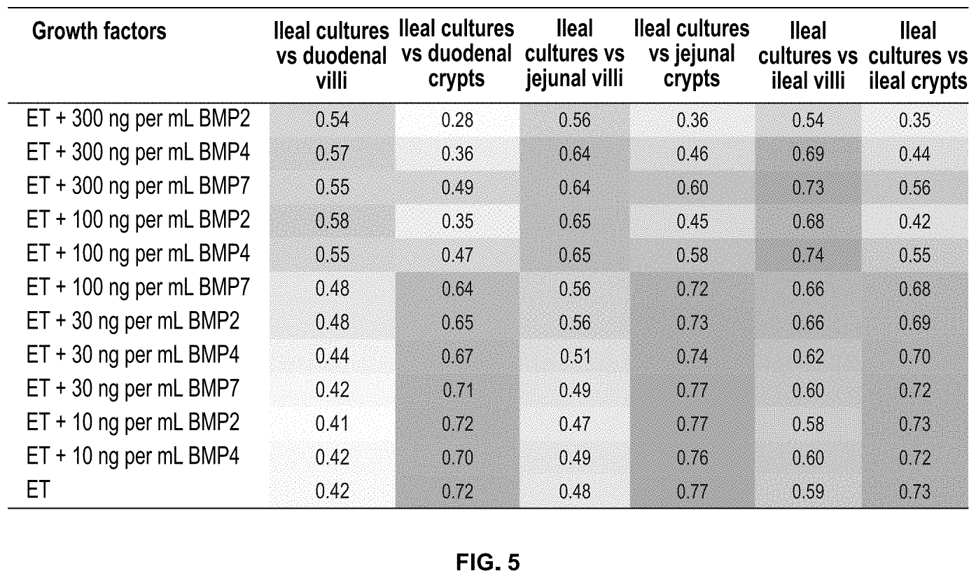

Dose titration of individual BMPs indicated that 100 ng/mL BMP 2 and 300 ng/mL BMP 4 resulted in cultures that had strong correlations of gene expression with tissues in the jejunum and ileum (FIG. 2B and FIG. 5). Further, BMP2 and BMP4 were as effective as the BMP mixture (i.e. BMP 2, 4, and 7) at differentiating mouse duodenal, jejunal, and ileal cultures (FIG. 2C). 300 ng/mL BMP 4, combined with EGF (E) and thiazovivin (T) was selected as the murine small intestine differentiation media (ET+BMP). The PCCs for small intestinal cultures were highest for the segment from which the cultures were derived (FIG. 2C), and many of the genes that showed segment-specific expression in intestinal tissue showed similar expression patterns in the cultures (FIG. 2D). Immunostaining for cell-specific markers confirmed that cells from absorptive, goblet, enteroendocrine, and Paneth cell lineages were present in these cultures (FIG. 2E).

Ion-transport analysis of Na⁺, K⁺, Cl⁻, and phosphate showed net Na⁺ absorption and apically-directed acid secretion in the small intestinal cultures, consistent with NHE3 expression throughout the small intestine (FIG. 2F-2G). Phosphate was actively absorbed only in the ileal culture, consistent with the exclusive expression of the Na*/phosphate co-transporter NaPi2b in the mouse ileum (28) (FIG. 2G-2H). Transport of K⁺ and Cl⁻ was unclear because the ion concentration was higher on the apical side than the basolateral side, suggesting net ion secretion, but when the change in apical volume was accounted for (apical volume decreases with time), both K⁺ and Cl⁻ showed net absorption (FIG. 2H). Therefore, the relative contributions of active, transcellular transport versus paracellular transport via diffusion and water drag were difficult to discern for K⁺ and Cl⁻ in the mouse small intestinal cultures.

### Example 4—Characterization of Human Intestinal Monolayer Cultures

As the availability of fresh human intestinal tissue is limited, human intestinal monolayer cultures were developed using cells derived from three-dimensional human intestinal organoids established from biopsies obtained from the duodenum, terminal ileum, and ascending, transverse, descending, and sigmoid colon according to a protocol approved by an institutional review board. Briefly, human biopsies were placed in DPBS after excision from the subject. Biopsy samples were then transferred to HypoThermosol FRS tissue-preservation media (BioLife Solutions) containing penicillin/streptomycin and stored on ice before being prepared for culture.

Biopsies were treated with TrypLE Express (Invitrogen), with 20 μM Y-27632 added, for 3 minutes at ambient temperature with intermittent pipetting. 6 mL of BM was added to quench the reaction and the sample was then centrifuged at 600×g for 3 minutes. The pellet was resuspended in 8 mL of BM. To separate slower-settling cells from faster-settling fragments of connective tissue, the media was collected using a 10 mL serological pipette as the connective tissue settled to the bottom of the tube. The collected media contained mostly single cells or cell clumps and was placed in a 15 mL conical centrifuge tube. The sample was centrifuged, and the pellet was resuspended in 250 μL ice-cold Matrigel. The Matrigel-cell suspension was quickly pipetted into a 24-well plate (Greiner Bio One) at 50 μL per well to form Matrigel domes. Organoid culture required the addition of nicotinamide (Nic), the TGFβ-R1 signaling inhibitor, A83-01 (A), and the p38 inhibitor, SB202190 (S), to WENR media to produce an organoid culture medium (WENRNicAS) for long-term growth (12). The Matrigel plate was placed in an incubator at 37° C., 5% CO₂ and allowed to solidify for approximately 10-20 minutes, and then 500 μL/well of SBM containing WENRNicASY with the GSK3 inhibitor, CHIR99021 (2.5 μM, Tocris), was added. Media changes were performed every Monday, Wednesday, and Friday using SBM containing WENRNicAS. CHIR99021 was added for only the first 2 days following cell isolation from the biopsy and was not added at any other stage. The cultures required passaging every 7-12 days and were typically split in a 1:6-1:8 ratio.

To passage the three-dimensional human organoid cultures, the Matrigel domes were broken apart in growth media and pooled in a 15 mL conical tube. The tube was centrifuged at 600×g for 3 minutes to pellet the cells and Matrigel. The pellet was resuspended in TrypLE Express and continuously pipetted for 4 minutes. To quench the reaction, 5 mL BM was added, and the sample was centrifuged at 600×g for 3 minutes to pellet the cells. The pellet was resuspended in the appropriate volume of Matrigel, plated at 50 μL per well in a 24-well plate, placed in an incubator at 37° C., 5% CO₂ for 10-20 minutes to solidify, and then 500 μL of SBM containing WENRNicASY was added to each well. Media changes for established cultures were performed every Monday, Wednesday, and Friday using WENRNicAS. Descriptions of media and solutions used for human intestinal organoid cultures are described in Table 3 below.

TABLE 3
Medias and Buffers for Human 3D Intestinal Organoid Cultures

| Solution Type | Component |
|---|---|
| Basal Media (BM) | Advanced DMEM/F12 |
| | 10 mM HEPES |
| | 1:100 Glutamax |
| | 1:100 Penicillin/Streptomycin |
| Supplemented Basal | 50 mL BM |
| Media (SBM) | 0.5 mL N2 Supplement |
| | 1 mL B27 Supplement |
| | 100 uL of 500 mM N-acetylcysteine |
| WENRNicASY with | SBM |
| CHIR99021 | 1:1000 of 50 μg/mL murine EGF |
| | 1:1000 of 100 μg/mL murine Noggin |
| | 1:1000 of 500 μg/mL human RSpondin-1 |
| | 1:500 of 50 μg/mL murine Wnt3a |
| | 1:100 of 1M Nicotinamide |
| | 1:2000 of 1 mM A83-01 |
| | 1:2000 of 20 mM SB202190 |
| | 1:10000 of 20 mM Y27632 |
| | 1:2000 of 5 mM CHIR99021 |
| WENRNicASY | SBM |
| | 1:1000 of 50 μg/mL murine EGF |
| | 1:1000 of 100 μg/mL murine Noggin |
| | 1:1000 of 500 μg/mL human RSpondin-1 |
| | 1:500 of 50 μg/mL murine Wnt3a |
| | 1:100 of 1M Nicotinamide |
| | 1:2000 of 1 mM A83-01 |
| | 1:2000 of 20 mM SB202190 |
| | 1:10000 of 20 mM Y27632 |
| WENRNicAS | SBM |
| | 1:1000 of 50 μg/mL murine EGF |
| | 1:1000 of 100 μg/mL murine Noggin |
| | 1:1000 of 500 μg/mL human RSpondin-1 |
| | 1:500 of 50 μg/mL murine Wnt3a |
| | 1:100 of 1M Nicotinamide |
| | 1:2000 of 1 mM A83-01 |
| | 1:2000 of 20 mM SB202190 |

Organoids were typically grown for 7-12 days before being used to plate monolayer cultures. Organoid cultures embedded in Matrigel were broken apart in growth media and pooled in a 15 mL conical tube. The cells were centrifuged at 600×g for 3 minutes and resuspended in TrypLE Express and placed in a water bath at 37° C. for 3 minutes. The cells were then removed and pipetted up and down for 7 minutes. BM was added to quench the reaction, and the cell suspension was centrifuged at 600×g for 3 minutes, resuspended in BM, and counted.

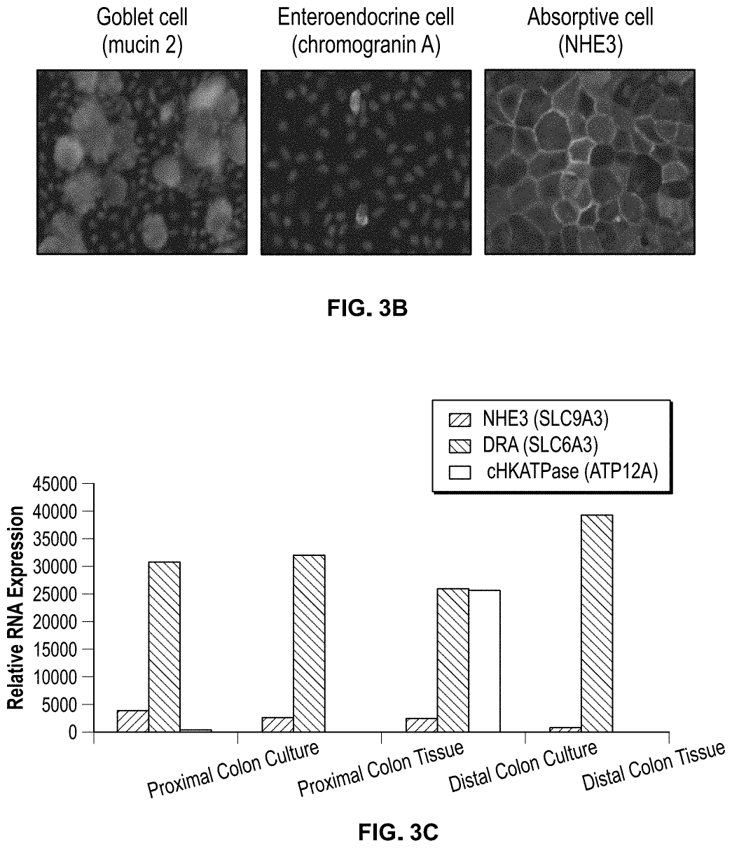

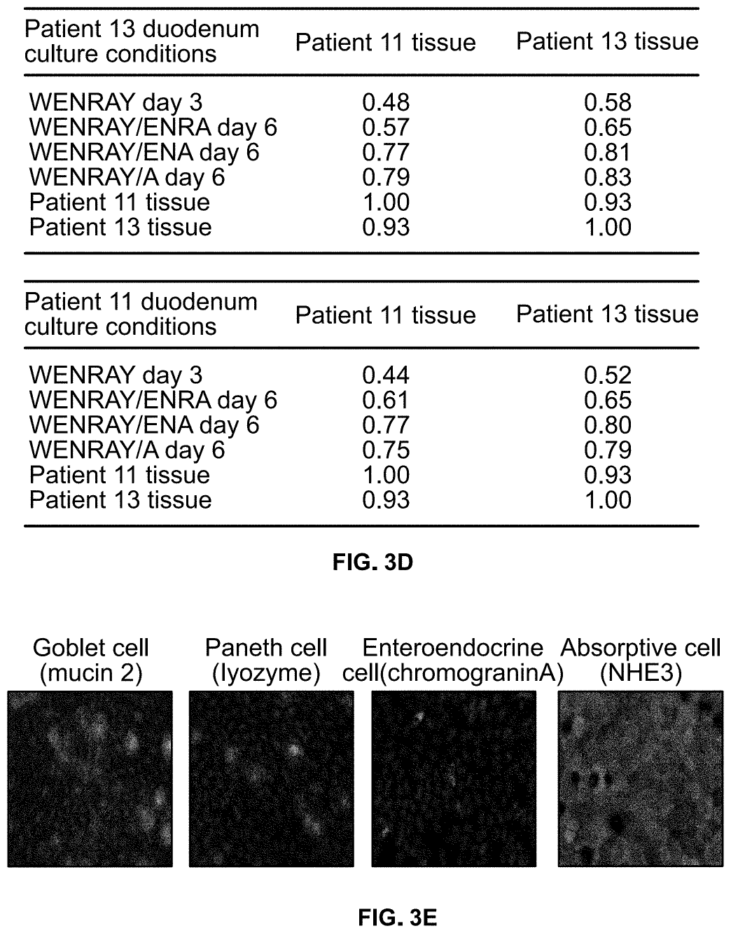

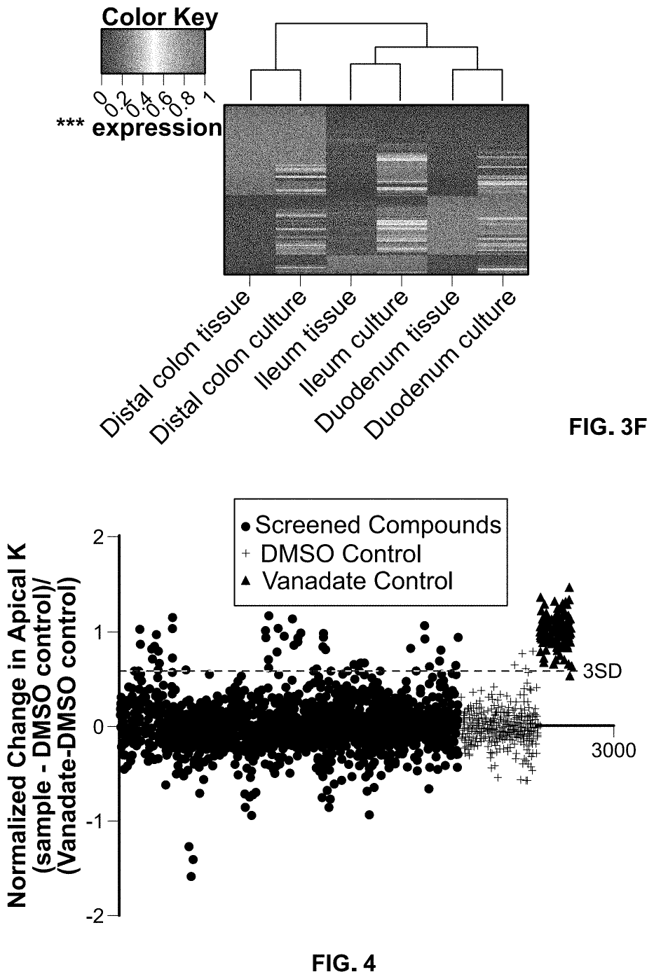

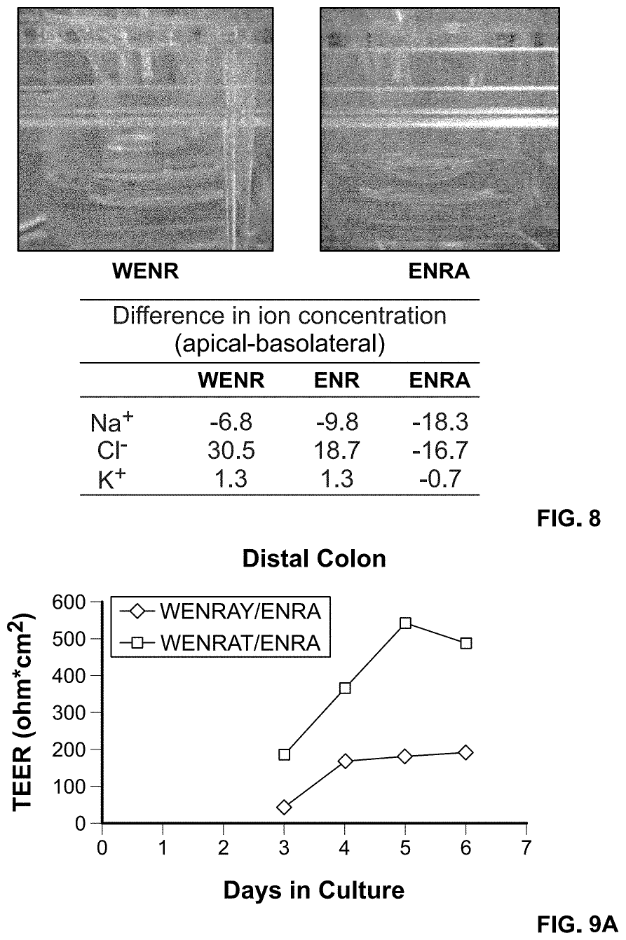

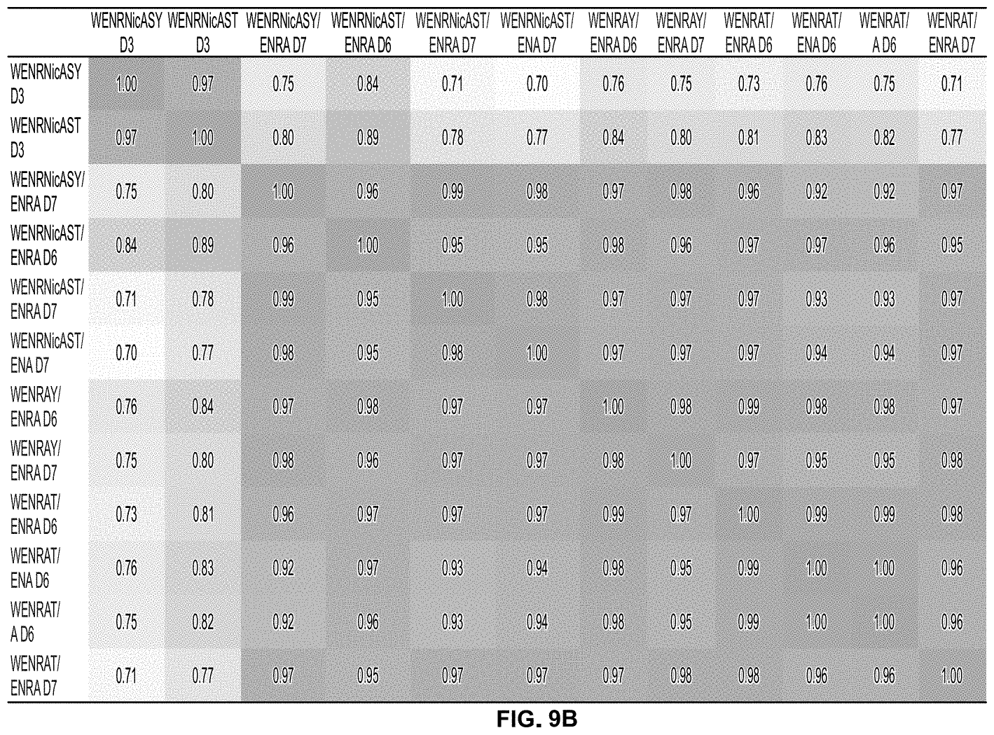

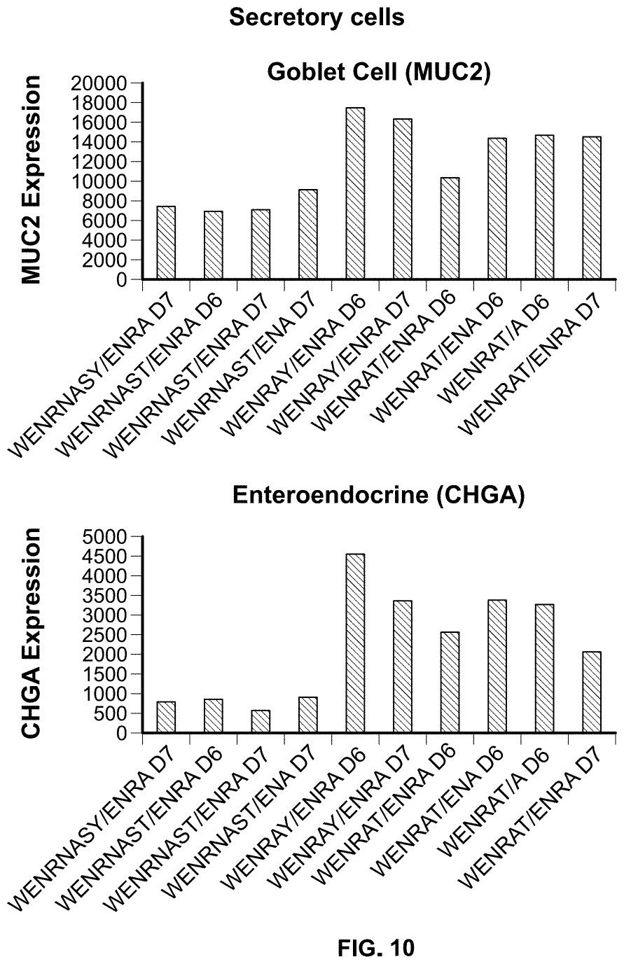

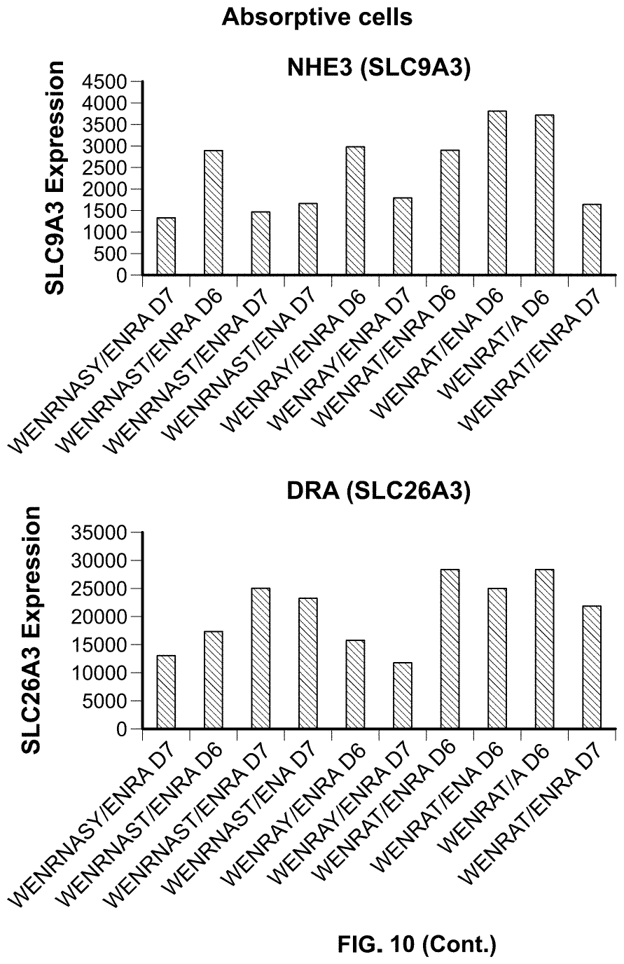

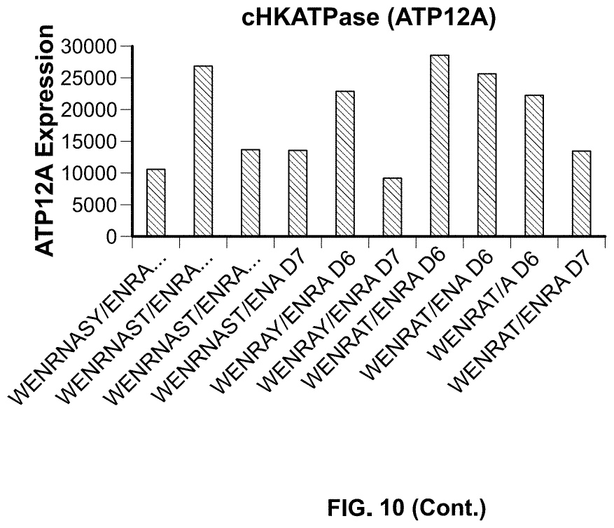

Media conditions used for murine colon growth and differentiation were optimized for human distal colon cultures using the water-absorption phenotype characteristic of this segment. The effects of seeding and differentiation media alterations on human distal colon cultures were determined by comparing two seeding medias (WENRAT or WENRNicAST) and three differentiation medias (WENR, ENRA, and ENR). Substitution of Y-27632 (Y) with thiazovivin (T) in the seeding media to generate WENRAT resulted in human colonic cultures that had higher TEER values while maintaining similar global transcriptional profiles, although thiazovivin resulted in some suppression of secretory cell differentiation (FIG. 9A-9B). Murine colonic growth media (WENR) was altered by removing Wnt3a (W) to generated ENR media, or by removing Wnt3a (W) and adding A83-01 (A) (ENRA). Removal of Wnt3a and addition of A83-01 was necessary for proper differentiation of water-absorbing cultures (FIG. 9B and FIG. 8). Human distal colonic cultures seeded in WENRAT or WENRNicAST and differentiated with ENRA, ENA, or A83-01 consistently yielded absorptive cultures that showed active transport of Na⁺, K⁺, and Cl⁻ (FIG. 3A and FIG. 9B), and global RNA sequencing indicated that cultures seeded with WENRAT or WENRNicAST seeding media were strongly correlated (FIG. 9B). However, the inclusion of Nic and SB202190 in the WENRNicAST seeding media further suppressed differentiation of secretory cells in human colonic cultures (FIG. 10). Therefore, seeding with WENRAT (human colonic seeding media) followed by differentiation with ENRA (human colonic differentiation media) was chosen as the standard media combination.

Immunostaining for cell lineage markers showed that all major cell types of the colon were present in the human cultures (FIG. 3B). Analysis of ion-transporter expression showed patterns similar to those observed in the mouse, with NHE3 and DRA expressed in both proximal and distal colonic cultures and colonic H⁺/K⁺-ATPase (ATP12A) expression restricted to the distal colonic cultures (FIG. 3C). While NHE3 and DRA expression levels in the cultures were similar to those in tissue, colonic H⁺/K⁺-ATPase expression was much higher in distal colonic cultures than in human distal colonic tissue (FIG. 3C).

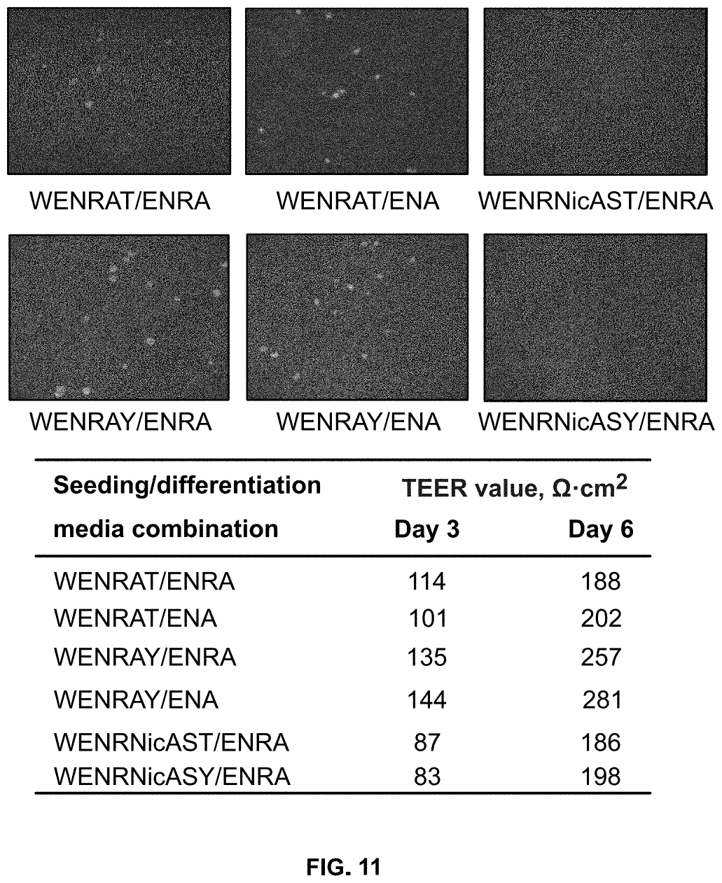

Initial attempts at culturing human duodenal and ileal tissues focused on seeding in WENRAY, WENRAT, WENRNicASY, and WENRNicAST media, and differentiating with ENRA. However, duodenal cultures seeded in WENRNicASY and WENRNicAST did not contain any goblet cells, and those seeded in WENRAT contained fewer goblet cells than those seeded in WENRAY (FIG. 11). Unlike the colonic cultures, no advantage in cell growth or TEER values resulted from substitution of Y-27632 with thiazovivin in either duodenal (FIG. 11) or ileal cultures. Therefore, further optimization of growth conditions focused on seeding cultures in WENRAY.

Similar to the strategy used for mouse cultures, global gene expression of human duodenal cultures was compared with that of duodenal tissue in order to enhance growth conditions. The Pearson correlation coefficient for gene expression of duodenal cultures seeded in WENRAY and differentiated with ENRA compared with that of duodenal tissue was highest when RNA was harvested from cultures on day 6, compared with days 3, 8, and 10 (FIG. 4). Moreover, duodenal cultures seeded in WENRAY and differentiated with either ENA or A83-01 showed better correlation with duodenal tissue than cultures differentiated with ENRA (FIG. 3D). Therefore, the selected growth conditions for human small intestinal cultures were WENRAY as the seeding media and ENA as the differentiation media.

Immunostaining for absorptive, goblet, enteroendocrine, and Paneth cell lineages confirmed that all of these major small intestinal cell lineages were present in the culture monolayer (FIG. 3E). Many of the most differentially expressed genes observed in comparisons of duodenal, ileal, and distal colonic tissue also showed consistent differential expression in the duodenal, ileal, and distal colonic cultures, indicating that the cultures maintained segment-specific gene expression patterns (FIG. 3F).

The optimized media conditions for human colon and small intesting monolayer cultures are summarized in Table 4.

TABLE 4
Medias and Buffers for Human 2D Monolayer Intestinal Cultures

| Solution Type | Component |
|---|---|
| Basal Media (BM) | Advanced DMEM/F12 |
| | 10 mM HEPES |
| | 1:100 Glutamax |
| | 1:100 Penicillin/Streptomycin |
| Supplemented Basal | 50 mL BM |
| Media (SBM) | 0.5 mL N2 Supplement |
| | 1 mL B27 Supplement |
| | 100 μL of 500 mM N-acetylcysteine |
| | 50 μL of 10 μM [Leu15]-Gastrin 1 |
| Human colon seeding | SBM |
| and growth media | 1:1000 of 50 μg/mL murine EGF |
| (WENRAT) | 1:1000 of 100 μg/mL murine Noggin |
| | 1:1000 of 500 μg/mL human RSpondin-1 |
| | 1:200 of 50 μg/mL murine Wnt3a |
| | 1:2000 of 1 mM A83-01 |
| | 1:2000 of 5 mM thiazovivin |
| Human small intestine | SBM |
| seeding and growth | 1:1000 of 50 μg/mL murine EGF |
| media (WENRAY) | 1:1000 of 100 μg/mL murine Noggin |
| | 1:1000 of 500 μg/mL human RSpondin-1 |
| | 1:200 of 50 μg/mL murine Wnt3a |
| | 1:2000 of 1 mM A83-01 |
| | 1:1000 of 20 mM Y-27632 |
| Human colon | SBM |
| differentiation | 1:1000 of 50 μg/mL murine EGF |
| media (ENRA) | 1:1000 of 100 μg/mL murine Noggin |
| | 1:1000 of 500 μg/mL human RSpondin-1 |
| | 1:2000 of 1 mM A83-01 |
| Human small intestine | SBM |
| differentiation | 1:1000 of 50 μg/mL murine EGF |
| media (ENA) | 1:1000 of 100 μg/mL murine Noggin |
| | 1:2000 of 1 mM A83-01 |
| Organoid Dissociation | TrypLE Express |
| Buffer | |

### Example 5—Compound Screen with Murine Distal Colon Monolayer Cultures

To demonstrate the utility of intestinal organoids grown as monolayers for phenotype screening, murine distal colonic monolayer cultures were scaled from 24-well to 96-well plates. Approximately 2,000 pharmacologically active compounds, covering a range of biological targets, were screened for their ability to block K⁺ absorption in mouse distal colonic cultures. Compounds screened comprised those from the LOPAC1280 library and the Tocriscreen library; several inhibitors of Na⁺/K⁺-ATPase were also screened owing to the homology of this enzyme with colonic H⁺/K⁺-ATPase. While K⁺ absorption in the distal colonic cultures was the primary readout, TEER and apically applied rubidium were used to counter-screen for compounds that block K⁺ absorption via the loss of tight junction integrity (thereby preventing the cultures from establishing a K⁺ gradient), rather than via inhibition of active K⁺ transport. Vanadate has been previously reported as a colonic H⁺/K⁺-ATPase inhibitor (Kaunitz J D et al, (1986), J Biol Chem 261(30):14005-14010), and 200 μM vanadate was able to block K⁺ absorption completely in the mouse distal colonic cultures. Vanadate was therefore chosen as a positive control to be included on each plate. Compounds were screened at a concentration of 30 μM.

Inhibition of K⁺ absorption was normalized to the mean value of vanadate controls on each plate such that a value of 1 indicated inhibition equivalent to vanadate. The mean±SD inhibition for all compounds screened was 0.02±0.25, for the DMSO negative controls was 0.00±0.17, and for the vanadate controls was 1.00±0.15. Compounds that inhibited K⁺ absorption to a degree greater than 3 SDs of the DMSO control (i.e. 0.50) were considered positive (FIG. 4). Compounds that inhibited K⁺ absorption but reduced TEER and showed increased rubidium transport owing to paracellular leakage were discarded.

1. A method for generating a two-dimensional (2D) monolayer cell culture of primary intestinal cells comprising the steps of:
(a) isolating cells from a mammalian tissue sample, wherein the tissue sample is a small intestine or a colon tissue sample;
(b) plating the cells in a monolayer in a well in the presence of a seeding medium, wherein the seeding medium comprises epidermal growth factor (EGF), a bone morphogenic protein (BMP) inhibitor, a leucine-rich repeat-containing G-protein coupled receptor (LGR)-5 activator, a Wnt signaling agonist, and a Rho-associated protein kinase (ROCK) inhibitor;
(c) growing the cells to a confluent monolayer in a growth medium; and
(d) differentiating the cells in a differentiation medium for a time sufficient for the cells to develop mature phenotype(s);
thereby generating a 2D monolayer cell culture of primary intestinal cells.

2. The method of claim 1, wherein the primary intestinal cells comprise one or more of enterocytes, goblet cells, enteroendocrine cells, Paneth cells, transit amplifying cells, and stem cells.

3. The method of claim 1, wherein the seeding medium, growth medium, and/or differentiation medium further comprise a growth promoting and/or an antioxidant factor.

4. The method claim 3, wherein the growth promoting factor comprises an N2 or B27 supplement.

5. The method of claim 3, wherein the antioxidant factor comprises N-acetylcysteine.

6. The method of claim 1, wherein the seeding medium comprises a concentration of about 5-500 ng/mL of EGF.

7. The method of claim 1, wherein the BMP inhibitor is Noggin and is at a concentration of about 10 ng/mL to about 500 ng/mL.

8. The method of claim 1, wherein the LGR5 activator is R-spondin 1 and is at a concentration of about 50 ng/mL to about 2 μg/mL, or about 100 ng/mL to about 1000 ng/mL.

9. The method of claim 1, wherein the Wnt signaling agonist is Wnt3a and is at a concentration of about 20 ng/mL to about 1 μg/mL.

10. The method of claim 1, wherein the ROCK inhibitor is Y-27632 or thiazovivin.

11. The method of claim 10, wherein the seeding medium comprises a Y-27632 concentration of about 1 μM to about 100 μM, or a thiazovivin concentration of about 0.5 μM to about 25 μM.

12. The method of claim 1, wherein the seeding medium comprises B27, N2, N-acetylcysteine, EGF, Noggin, R-Spondin-1, Wnt3a, and Y-27632.

13. The method of claim 12, wherein the seeding medium comprises B27, N2, about 1 mM N-acetylcysteine, about 50 ng/mL EGF, about 0.1 μg/mL Noggin, about 250 ng/mL Wnt3a, about 0.5 μg/mL R-spondin 1, and about 20 μM Y27632.

14. The method of claim 1, wherein the growth medium comprises EGF, a BMP inhibitor, an LGR5 activator, and a Wnt signaling agonist.

15. (canceled)

16. The method of claim 14, wherein the BMP inhibitor is Noggin and is at a concentration of about 10 ng/mL to about 500 ng/mL.

17. The method of claim 14, wherein the LGR5 activator is R-spondin 1 and is at a concentration of about 50 ng/mL to about 2 μg/mL.

18-52. (canceled)

53. A method for generating a two-dimensional (2D) monolayer cell culture of stable, primary intestinal cells comprising:
(a) obtaining organoids, wherein the organoids are human intestinal organoids cultured from a human small intestine tissue sample or a human colon tissue sample;
(b) dissociating cells from the organoids;
(c) plating the cells in a monolayer in a well in the presence of a seeding culture medium, wherein the seeding culture medium comprises epidermal growth factor (EGF), a bone morphogenic protein (BMP) inhibitor, a leucine-rich repeat-containing G-protein coupled receptor (LGR)-5 activator, a Wnt signaling agonist, a transforming growth factor (TGF)-β signaling antagonist, and a ROCK inhibitor;
(d) growing the cells to a confluent monolayer in a growth medium; and
(e) differentiating cells in a differentiation medium for a time sufficient for the cells to develop mature phenotype(s).

54-126. (canceled)

127. A method of performing a high throughput screen, comprising performing a screen with a plurality of agents on a primary cell culture to identify agents within the plurality of agents that modifies a property of the primary cell culture, wherein the primary cell culture originates from intestine or colon, and wherein each agent of the plurality of agents is screened according to a methods of any one of the preceding claims.

128-203. (canceled)

204. A method for inhibiting the transport of sodium across the two-dimensional (2D) monolayer cell culture prepared according to the method of claim 1, comprising contacting the apical side of said monolayer with the compound

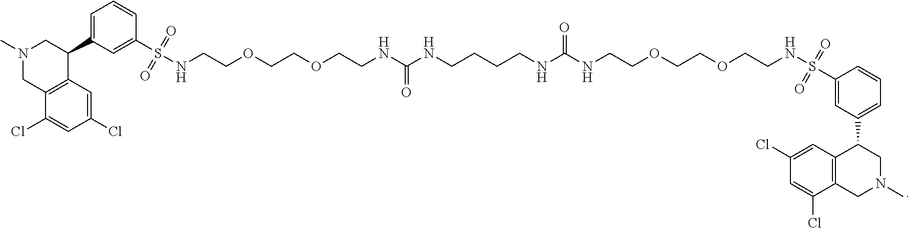

205. A method for inhibiting the transport of phosphate across a two-dimensional (2D) monolayer cell culture as described herein, comprising contacting the apical side of said monolayer with the compound 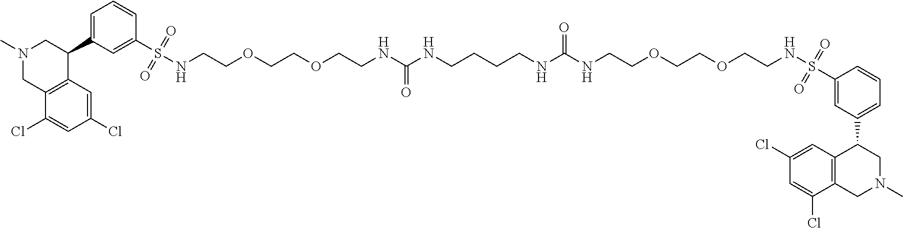
or a pharmaceutically acceptable salt thereof.
